(19) United States
(12) Patent Application Publication
(10) Pub. No.: US 2004/0009990 A1
(43) Pub. Date: Jan. 15, 2004

(54) Method to treat cystic fibrosis

(76) Inventors: Linda S. Higgins, Palo Alto, CA (US); David Y. Liu, Palo Alto, CA (US); Andrew A. Protter, Palo Alto, CA (US)

Correspondence Address: Kate H. Murashige, Morrison & Foerster LLP, Suite 500, 3811 Valley Centre Drive, San Diego, CA, 92130, US

(21) Appl. No.: 10/291,243

(22) Filed: Nov. 8, 2002

Related U.S. Application Data
(60) Provisional application No. 60/338,209, filed on Nov. 9, 2001.

(51) Int. Cl. A61K 31/496; A61K 31/454

(52) U.S. Cl. 514/254.06; 514/322; 514/323

(57) ABSTRACT

The invention is directed to methods to treat cystic fibrosis by administering certain imidazole derivatives.

### TECHNICAL FIELD

The invention is directed to a method to treat cystic fibrosis using indole derivatives. This application claims priority to U.S. App. No. 60/338,209, filed Nov. 9, 2001, incorporated by reference herein.

### BACKGROUND ART

PCT publication WO00/71535 published Dec. 7, 2000 discloses indole derived compounds that are specific inhibitors of p38 kinase α. The disclosure of this document is incorporated herein by reference. It is disclosed in that document that inhibitors of the kinase activity of p38-α are useful anti-inflammatory agents. It is further understood that p38 mitogen activated protein kinase (p38-MAPK) plays a role in pulmonary inflammation.

More specifically, a paper by Nick, J. A., et al., J. Immunol. (2000) 164:2151-2159 describes a murine model of mild LPS induced lung inflammation. It had been shown in vitro that exposure to an inhibitor of p38-MAPK blocks TNF-α and macrophage inflammatory protein 2 (MIP-2) release from murine and human neutrophils and macrophage and eliminates migration of murine neutrophils toward the chemokines MIP-2 and KC. In contrast, alveolar macrophage required a thousand-fold greater concentration of the inhibitor to block release of TNF-α and MIP-2 in the mouse model itself, inhibition of p38-MAPK decreased the release of TNF-α and neutrophil accumulation in air spaces, but recovery of MIP-2 and KC from air spaces was not affected by this. Also accumulation of mononuclear cells was not significantly reduced. The authors conclude that the greater dependence by neutrophils when compared to other leukocytes on p38-MAPK cascades suggests a method to modulate early inflammation in the lung.

Underwood, D. C., et al., Am. J. Physiol. Lung Cell Mol. Physiol. (2000) 279:L895-L902 studied the effects of a p38-MAPK kinase inhibitor in murine models of chronic obstructive pulmonary disease and in a model of lung fibrosis. They found that airway neutrophil infiltration and IL-6 levels were decreased by administration of the inhibitor in a bleomycin induced pulmonary fibrosis model in rats. The inhibitor depleted right ventricular hypertrophy which is indicative of secondary pulmonary hypertension. The authors concluded that the inhibitor is effective against a range of sequelae commonly associated with chronic obstructive pulmonary disease and fibrosis.

In addition, Loitsch, S. M., et al., Biochem. Biophys. Res. Commun. (2000) 276:571-578 in in vitro studies using bronchial epithelial cells concluded that p38-MAPK inhibitors reduced hyperosmolarity-induced IL-8 synthesis. Antioxidants were shown to block the activation of p38-MAPK that is induced by hyperosmolarity.

PCT publication WO99/19473 speculates that inhibitors of p38 (and a multiplicity of other proteins) may be useful in treating cardiac hypertrophy. This document further speculates that among cardiac hypertrophy induced dysfunctions may be included cystic fibrosis.

The foregoing documents are exemplary of the general knowledge that p38-α kinase or p38-MAPK inhibitors exert anti-inflammatory effects and reduce neutrophil migration.

Reddi, K., et al., FASEB Journal (2001) 15:A588 disclose that an inhibitor of p38 kinase inhibits the secretion of IL-8 by human lung epithelial cells after infection of these cells with B. cepacia, which is stated to be a prevalent pulmonary pathogen in cystic fibrosis.

Cystic fibrosis itself is known to be the result of a genetic defect in a gene which encodes a chloride ion channel. The chloride ion channel must be present in active form in order to prevent plugging secretory ducts in various tissues, most importantly in lung, but also in the pancreas and in the reproductive organs of the male. Because the secretory ducts are plugged, mucus tends to accumulate in these organs, and the organs, especially the lung, become targets for infection which is difficult to control. The inflammatory responses and migration of neutrophils into the lungs of cystic fibrosis sufferers may be a response to this infection.

In general, cystic fibrosis is characterized by chronic lung inflammation including a massive infiltration of lung by neutrophils. The inflammation precedes bacterial or microbial infection and this infection is a major cause of morbidity and mortality. There is considerable mucus plugging and elastase and inflammatory mediators cause progressive damage.

Baudouin-Legros, M., et al., Am. J. Physiol. Cell Physiol. (2000) 278:C49-56 note the importance of the action of hypertonicity on cystic fibrosis gene expression. Cystic fibrosis transmembrane conductance regulator (CFTR) is the cAMP-regulated chloride channel which regulates ion transport across secretory epithelia. It is this gene which is defective in individuals with cystic fibrosis. Expression of this gene is decreased by added chloride ion, but this decrease requires p38 kinase cascade activity as shown by the effects of administering inhibitors of this enzyme. The authors note, however, the overall complexity of this process.

In summary, the effects of p38-α which have been established in the art include inhibition of chemotaxis but not chemokinesis of lung neutrophils; blockage of MIP-2 and TNF-α secretion by neutrophils; blockage of stress-induced apoptosis of neutrophils, inhibition of IL-8 secretion from bronchial epithelial cells; inhibition of stiffening of pulmonary microvascular endothelial cells; and reduction of neutrophil migration. Some of these observations have been verified in animal models where it has been shown that inhibitors of p38-α kinase attenuate the secretion of IL-6 and MMP-9 as well as TNF-α production by neutrophils.

It is also understood that lung macrophage are refractory to p38 inhibition, and in an additional study on P. aeruginosa, which is a persistent pathogen in the airways of patients with cystic fibrosis, Terada, L. S., et al., Infect. Immun. (1999) 67:2371-2376 suggest that control of this infection is mediated by pathways that are independent of p38-α kinase. CFTR mutant mice are hyper-responsive to Pseudomonas, so amelioration of cystic fibrosis would desirably involve control of this infection.

Current treatments of cystic fibrosis are not entirely satisfactory. High dose ibuprofen and dosages of prednisone, while efficacious, have unacceptable side effects, and although the Cystic Fibrosis Foundation recommends chronic ibuprofen treatment, less than 10% of patients are treated in this manner because of the side effects.

It is apparent that although it is understood that p38-α kinase is required for response to stimulants that mobilize neutrophil migration into the lung such as those found in disease states and thus the release of cytokines by the neutrophils, the ability of inhibitors of p38-α kinase to ameliorate the symptoms of, or successfully treat or prevent, cystic fibrosis is unclear. There is a multiplicity of mechanisms at work, and the complete inhibition of neutrophil migration would constitute an undesirable side effect of inhibiting the inflammatory response since the presence of the neutrophils is a major factor in controlling the infections attracted by the excess of mucus. Accordingly, the present invention resolves this ambiguity by providing a method to treat cystic fibrosis using certain derivatives of indole.

### DISCLOSURE OF THE INVENTION

The invention is directed to methods and compounds useful in treating cystic fibrosis in humans.

The compounds of the invention are of the formula

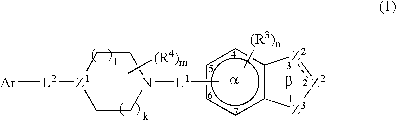

and the pharmaceutically acceptable salts thereof, or a pharmaceutical composition thereof, wherein

[symbol] represents a single or double bond;

one Z² is CA or CR⁸A and the other is CR¹, CR¹₂, NR⁶ or N wherein each R¹, R⁶ and R⁸ is independently hydrogen or noninterfering substituent;

A is -Wᵢ-COXⱼY wherein Y is COR² or an isostere thereof and R² is hydrogen or a noninterfering substituent, each of W and X is a spacer of 2-6 Å, and each of i and j is independently 0 or 1;

Z³ is NR⁷ or O;

R⁷ is a noninterfering substituent;

each R³ is independently a noninterfering substituent;

n is 0-3;

each of L¹ and L² is a linker;

each R⁴ is independently a noninterfering substituent;

m is 0-4;

Z¹ is CR⁵ or N wherein R⁵ is hydrogen or a noninterfering substituent;

each of l and k is an integer from 0-2 wherein the sum of l and k is 0-3;

Ar is an aryl group substituted with 0-5 noninterfering substituents, wherein two noninterfering substituents can form a fused ring; and

the distance between the atom of Ar linked to L² and the center of the α ring is 4.5-24 Å.

The invention is directed to methods of treating cystic fibrosis conditions using these compounds or pharmaceutical compositions thereof. The method comprises administering to a subject in need of such treatment an effective amount of the compound of formula (1) or a pharmaceutical composition thereof.

### MODES OF CARRYING OUT THE INVENTION

The Compounds of Formula (1) are Useful in Treating Cystic Fibrosis

The compounds useful in the invention are derivatives of indole-type compounds containing a mandatory substituent, A, at a position corresponding to the 2- or 3-position of indole. In general, an indole-type nucleus is preferred, although alternatives within the scope of the invention are also illustrated below.

In the description above, certain positions of the molecule are described as permitting “noninterfering substituents.” This terminology is used because the substituents in these positions generally speaking are not relevant to the essential activity of the molecule taken as a whole. A wide variety of substituents can be employed in these positions, and it is well within ordinary skill to determine whether any particular arbitrary substituent is or is not “noninterfering.”

As used herein, a “noninterfering substituent” is a substituent which leaves the ability of the compound of formula (1) to inhibit p38-α activity qualitatively intact. Thus, the substituent may alter the degree of inhibition of p38-α. However, as long as the compound of formula (1) retains the ability to inhibit p38-α activity, the substituent will be classified as “noninterfering.” A number of assays for determining the ability of any compound to inhibit p38-α activity are available in the art. A whole blood assay for this evaluation is illustrated below: the gene for p38-α has been cloned and the protein can be prepared recombinantly and its activity assessed, including an assessment of the ability of an arbitrarily chosen compound to interfere with this activity. The essential features of the molecule are tightly defined. The positions which are occupied by “noninterfering substituents” can be substituted by conventional organic moieties as is understood in the art. It is irrelevant to the present invention to test the outer limits of such substitutions. The essential features of the compounds are those set forth with particularity herein.

In addition, L¹ and L² are described herein as linkers. The nature of such linkers is less important that the distance they impart between the portions of the molecule. Typical linkers include alkylene, i.e. (CH₂)ₙ—R; alkenylene—i.e., an alkylene moiety which contains a double bond, including a double bond at one terminus. Other suitable linkers include, for example, substituted alkylenes or alkenylenes, carbonyl moieties, and the like.

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when so stated however, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, “inorganic residue” refers to a residue that does not contain carbon. Examples include, but are not limited to, halo, hydroxy, NO₂ or NH₂.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-1° C. (alkyl) or 2-1° C. (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2 O, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety refers to a monocyclic or fused bicyclic moiety such as phenyl or naphthyl; “heteroaromatic” also refers to monocyclic or fused bicyclic ring systems containing one or more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety.

When the compounds of Formula 1 contain one or more chiral centers, the invention includes optically pure forms as well as mixtures of stereoisomers or enantiomers

With respect to the portion of the compound between the atom of Ar bound to L and ring α, L¹ and L² are linkers which space the substituent Ar from ring a at a distance of 4.5-24 Å, preferably 6-20 Å, more preferably 7.5-10 Å. The distance is measured from the center of the α ring to the atom of Ar to which the linker L² is attached. Typical, but nonlimiting, embodiments of L¹ and L² are CO and isosteres thereof, or optionally substituted isosteres, or longer chain forms. L², in particular, may be alkylene or alkenylene optionally substituted with noninterfering substituents or L1 or L2 may be or may include a heteroatom such as N, S or O. Such substituents include, but are limited to, a moiety selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety.

Isosteres of CO and CH₂, include SO, SO₂, or CHOH. CO and CH₂ are preferred.

Thus, L² is substituted with 0-2 substituents. Where appropriate, two optional substituents on L² can be joined to form a non-aromatic saturated or unsaturated hydrocarbyl ring that includes 0-3 heteroatoms such as O, S and/or N and which contains 3 to 8 members. Two optional substituents on L2 can be joined to form a carbonyl moiety which can be subsequently converted to an oxime, an oximeether, an oximeester, or a ketal.

Ar is aryl, heteroaryl, including 6-5 fused heteroaryl, cycloaliphatic or cycloheteroaliphatic that can be optionally substituted. Ar is preferably optionally substituted phenyl.

Each substituent on Ar is independently a hydrocarbyl residue (1-20C) containing 0-5 heteroatoms selected from O, S and N, or is an inorganic residue. Preferred substituents include those selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two of said optional substituents on adjacent positions can be joined to form a fused, optionally substituted aromatic or nonaromatic, saturated or unsaturated ring which contains 3-8 members. More preferred substituents include halo, alkyl (1-4C) and more preferably, fluoro, chloro and methyl. These substituents may occupy all available positions of the aryl ring of Ar, preferably 1-2 positions, most preferably one position. These substituents may be optionally substituted with substituents similar to those listed. Of course some substituents, such as halo, are not further substituted, as known to one skilled in the art.

Two substituents on Ar can be joined to form a fused, optionally substituted aromatic or nonaromatic, saturated or unsaturated ring which contains 3-8 members.

Between L¹ and L² is a piperidine-type moiety of the following formula:

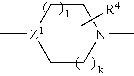

Z¹ is CR⁵ or N wherein R⁵ is H or a noninterfering substituent. Each of l and k is an integer from 0-2 wherein the sum of l and k is 0-3. The noninterfering substituents R⁵ include, without limitation, halo, alkyl, alkoxy, aryl, arylalkyl, aryloxy, heteroaryl, acyl, carboxy, or hydroxy. Preferably, R⁵ is H, alkyl, OR, NR₂, SR or halo, where R is H or alkyl. Additionally, R⁵ can be joined with an R⁴ substituent to form an optionally substituted non-aromatic saturated or unsaturated hydrocarbyl ring which contains 3-8 members and 0-3 heteroatoms such as O, N and/or S. Preferred embodiments include compounds wherein Z¹ is CH or N, and those wherein both l and k are 1.

R⁴ represents a noninterfering substituent such as a hydrocarbyl residue (1-20C) containing 0-5 heteroatoms selected from O, S and N. Preferably R⁴ is alkyl, alkoxy, aryl, arylalkyl, aryloxy, heteroalkyl, heteroaryl, heteroarylalkyl, RCO, ═O, acyl, halo, CN, OR, NRCOR, NR, wherein R is H, alkyl (preferably 1-4C), aryl, or hetero forms thereof. Each appropriate substituent is itself unsubstituted or substituted with 1-3 substituents. The substituents are preferably independently selected from a group that includes alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof. R⁴ may occur m times on the ring; m is an integer of 0-4. Preferred embodiments of R⁴ comprise alkyl (1-4C) especially two alkyl substituents and carbonyl. Most preferably R⁴ comprises two methyl groups at positions 2 and 5 or 3 and 6 of a piperidinyl or piperazinyl ring or ═O preferably at the 5-position of the ring. The substituted forms may be chiral and an isolated enantiomer may be preferred.

R³ also represents a noninterfering substituent. Such substituents include hydrocarbyl residues (1-6C) containing 0-2 heteroatoms selected from O, S and/or N and inorganic residues. n is an integer of 0-3, preferably 0 or 1. Preferably, the substituents represented by R³ are independently halo, alkyl, heteroalkyl, OCOR, OR, NRCOR, SR, or NR₂, wherein R is H, alkyl, aryl, or heteroforms thereof. More preferably R³ substituents are selected from alkyl, alkoxy or halo, and most preferably methoxy, methyl, and chloro. Most preferably, n is 0 and the a ring is unsubstituted, except for L¹ or n is 1 and R³ is halo or methoxy.

In the ring labeled β, Z³ may be NR⁷ or O—i.e., the compounds may be related to indole or benzofuran. If C³ is NR⁷, preferred embodiments of R⁷ include H or optionally substituted alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof. More preferably, R⁷ is hydrogen or is alkyl (1-4C), preferably methyl or is acyl (1-4C), or is COOR wherein R is H, alkyl, alkenyl of aryl or hetero forms thereof. R⁷ is also preferably a substituted alkyl wherein the preferred substituents are form ether linkages or contain sulfinic or sulfonic acid moieties. Other preferred substituents include sulfhydryl substituted alkyl substituents. Still other preferred substituents include CONR₂ wherein R is defined as above.

It is preferred that the indicated dotted line represents a double bond; however, compounds which contain a saturated β ring are also included within the scope of the invention.

Preferably, the mandatory substituent CA or CR⁸A is in the 3-position; regardless of which position this substituent occupies, the other position is CR¹, CR¹₂, NR⁶ or N. CR¹ is preferred. Preferred embodiments of R¹ include hydrogen, alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂ CN, CF₃, R₃Si, and NO₂, wherein each R is independently H, alkyl, alkenyl or aryl or heteroforms thereof and two of R¹ can be joined to form a fused, optionally substituted aromatic or nonaromatic, saturated or unsaturated ring which contains 3-8 members. Most preferably, R¹ is H, alkyl, such as methyl, most preferably, the ring labeled a contains a double bond and CR¹ is CH or C-alkyl. Other preferable forms of R¹ include H, alkyl, acyl, aryl, arylalkyl, heteroalkyl, heteroaryl, halo, OR, NR₂, SR, NRCOR, alkyl-OOR, RCO, COOR, and CN, wherein each R is independently H, alkyl, or aryl or heteroforms thereof.

While the position not occupied by CA is preferred to include CR¹, the position can also be N or NR⁶. While NR⁶ is less preferred (as in that case the ring labeled β would be saturated), if NR⁶ is present, preferred embodiments of R⁶ include H, or alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, or R₃Si wherein each R is independently H, alkyl, alkenyl or aryl or heteroforms thereof.

Preferably, CR⁸A or CA occupy position 3—and preferably Z² in that position is CA. However, if the β ring is saturated and R⁸ is present, preferred embodiments for R⁸ include H, halo, alkyl, alkenyl and the like. Preferably R is a relatively small substituent corresponding, for example, to H or lower alkyl 1-4C.

A is -Wᵢ-COXⱼY wherein Y is COR² or an isostere thereof and R² is a noninterfering substituent. Each of W and X is a spacer and may be, for example, optionally substituted alkyl, alkenyl, or alkynyl, each of i and j is 0 or 1. Preferably, W and X are unsubstituted. Preferably, j is 0 so that the two carbonyl groups are adjacent to each other. Preferably, also, i is 0 so that the proximal CO is adjacent the ring. However, compounds wherein the proximal CO is spaced from the ring can readily be prepared by selective reduction of an initially glyoxal substituted β ring. In the most preferred embodiments of the invention, the α/β ring system is an indole containing CA in position 3—and wherein A is COCOR².

The noninterfering substituent represented by R², when R² is other than H, is a hydrocarbyl residue (1-20C) containing 0-5 heteroatoms selected from O, S and/or N or is an inorganic residue. Preferred are embodiments wherein R² is H, or is straight or branched chain alkyl, alkenyl, alkynyl, aryl, arylalkyl, heteroalkyl, heteroaryl, or heteroarylalkyl, each optionally substituted with halo, alkyl, heteroalkyl, SR, OR, NR₂, OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroatom-containing forms thereof, or wherein R² is OR, NR₂, SR, NRCONR₂, OCONR₂, or NRSO₂NR₂, wherein each R is independently H, alkyl, alkenyl or aryl or the heteroatom-containing forms thereof, and wherein two R attached to the same atom may form a 3-8 member ring and wherein said ring may further be substituted by alkyl, alkenyl, alkynyl, aryl, arylalkyl, heteroalkyl, heteroaryl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroatom-containing forms thereof wherein two R attached to the same atom may form a 3-8 member ring, optionally substituted as above defined.

Other preferred embodiments of R² are H, heteroarylalkyl, —NR₂, heteroaryl, —COOR, —NHRNR₂, heteroaryl-COOR, heteroaryloxy, —OR, heteroaryl-NR₂, —NROR and alkyl. Most preferably R² is isopropyl piperazinyl, methyl piperazinyl, dimethylamine, piperazinyl, isobutyl carboxylate, oxycarbonylethyl, morpholinyl, aminoethyldimethylamine, isobutyl carboxylate piperazinyl, oxypiperazinyl, ethylcarboxylate piperazinyl, methoxy, ethoxy, hydroxy, methyl, amine, aminoethyl pyrrolidinyl, aminopropanediol, piperidinyl, pyrrolidinyl-piperidinyl, or methyl piperidinyl.

Isosteres of COR² as represented by Y are defined as follows.

The isosteres have varying lipophilicity and may contribute to enhanced metabolic stability. Thus, Y, as shown, may be replaced by the isosteres in Table 1.

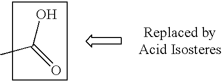

TABLE 1
Acid Isosteres

| Names of Groups | Chemical Structures | Substitution Groups (SG) |
|---|---|---|
| tetrazole | 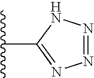 | n/a |
| 1,2,3-triazole | 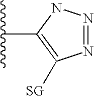 | H; SCH₃; COCH₃; Br; SOCH₃; SO₂CH₃; NO₂; CF₃; CN; COOMe |
| 1,2,4-triazole | 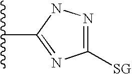 | H; SCH₃; COCH₃; Br; SOCH₃; SO₂CH₃; NO₂ |
| imidazole | 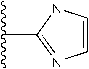 | H; SCH₃; COCH₃; Br; SOCH₃; SO₂CH₃; NO₂ |

Thus, isosteres include tetrazole, 1,2,3-triazole, 1,2,4-triazole and imidazole.

The compounds of formula (1) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1), the compound may also be supplied as a salt with a pharmaceutically acceptable cation.

The compounds of the invention may also be supplied in a prodrug form. Where chiral centers exist by virtue of the substituents in the compounds of the invention, individual stereoisomers or mixtures of stereoisomers may be used in the methods of the invention.

Utility and Administration

The methods and compositions of the invention are successful to treat or ameliorate cystic fibrosis in humans.

As used herein, “treat” or “treatment” include effecting postponement of development of undesirable conditions and/or reduction in the severity of such symptoms that will or are expected to develop. Treatment includes ameliorating existing symptoms, preventing additional symptoms, ameliorating or preventing the underlying metabolic causes of symptoms, preventing the severity of the condition or reversing the condition, at least partially. Thus, the terms denote that a beneficial result has been conferred on a subject with cystic fibrosis.

Treatment generally comprises “administering” a subject compound which includes providing the subject compound in a therapeutically effective amount. “Therapeutically effective amount” means the amount of the compound that will treat cystic fibrosis by eliciting a favorable response in a cell, tissue, organ, system, in a human. The response may be preventive or therapeutic. The administering may be of the compound per se in a pharmaceutically acceptable composition, or this composition may include combinations with other active ingredients that are suitable to the treatment of this condition. The compounds may be administered in a prodrug form.

The manner of administration and formulation of the compounds useful in the invention and their related compounds will depend on the nature of the condition, the severity of the condition, the particular subject to be treated, and the judgement of the practitioner; formulation will also depend on mode of administration. As the compounds of the invention are “small molecules,” they are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of 5%-95% of the total formulation, but wide variation is permitted depending on the carrier. Suitable carriers include sucrose, pectin, magnesium stearate, lactose, peanut oil, olive oil, water, and the like. This method is preferred if the subject can tolerate oral administration. Severe cystic fibrosis impairs gut absorption and metabolism so that it may not be possible to use this route when the condition is advanced.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, for topical conditions such as psoriasis, or in formulation intended to penetrate the skin. These include lotions, creams, ointments and the like which can be formulated by known methods.

The compounds may also be administered by injection, including intravenous, intramuscular, subcutaneous or intraperitoneal injection. Typical formulations for such use are liquid formulations in isotonic vehicles such as Hank's solution or Ringer's solution.

Intravenous administration is preferred for acute conditions; generally in these circumstances, the subject will be hospitalized. The intravenous route avoids any problems with inability to absorb the orally administered drug.

Alternative formulations include nasal sprays, liposomal formulations, slow-release formulations, and the like, as are known in the art. As cystic fibrosis severely affects the lungs, delivery via nebulizer, inhaler and otherwise directly into the lungs is also a preferred route of administration as the effects are relatively localized.

Any suitable formulation may be used. A compendium of art-known formulations is found in Remington's Pharmaceutical Sciences, latest edition, Mack Publishing Company, Easton, Pa. Reference to this manual is routine in the art.

Thus, the compounds useful in the method of the invention may be administered systemically or locally. For systemic use, the compounds are formulated for parenteral (e.g., intravenous, subcutaneous, intramuscular, intraperitoneal, intranasal or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three times daily. Alternatively, the compounds may be administered in a cyclical manner (administration of compound; followed by no administration; followed by administration of compound, and the like). Treatment will continue until the desired outcome is achieved. In general, pharmaceutical formulations will include an active ingredient in combination with a pharmaceutically acceptable vehicle, such as saline, buffered saline, 5% dextrose in water, borate-buffered saline containing trace metals or the like. Formulations may further include one or more excipients, preservatives, solubilizers, buffering agents, albumin to prevent protein loss on vial surfaces, lubricants, fillers, stabilizers, etc.

Pharmaceutical compositions can be in the form of sterile, non-pyrogenic liquid solutions or suspensions, coated capsules, suppositories, lyophilized powders, transdermal patches or other forms known in the art.

Biodegradable films or matrices may be used in the invention methods. These include calcium sulfate, tricalcium phosphate, hydroxyapatite, polylactic acid, polyanhydrides, bone or dermal collagen, pure proteins, extracellular matrix components and the like and combinations thereof. Such biodegradable materials may be used in combination with non-biodegradable materials, to provide desired mechanical, cosmetic or tissue or matrix interface properties.

Alternative methods for delivery may include osmotic minipumps; sustained release matrix materials such as electrically charged dextran beads; collagen-based delivery systems, for example; methylcellulose gel systems; alginate-based systems, and the like.

Aqueous suspensions may contain the active ingredient in admixture with pharmacologically acceptable excipients, comprising suspending agents, such as methyl cellulose; and wetting agents, such as lecithin, lysolecithin or long-chain fatty alcohols. The said aqueous suspensions may also contain preservatives, coloring agents, flavoring agents, sweetening agents and the like in accordance with industry standards.

Preparations for topical and local application comprise aerosol sprays, lotions, gels and ointments in pharmaceutically appropriate vehicles which may comprise lower aliphatic alcohols, polyglycols such as glycerol, polyethylene glycol, esters of fatty acids, oils and fats, and silicones. The preparations may further comprise antioxidants, such as ascorbic acid or tocopherol, and preservatives, such as p-hydroxybenzoic acid esters.

Parenteral preparations comprise particularly sterile or sterilized products. Injectable compositions may be provided containing the active compound and any of the well known injectable carriers. These may contain salts for regulating the osmotic pressure.

Liposomes may also be used as a vehicle, prepared from any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine, or phosphatidylinositol and the like. Synthetic phospholipids may also be used.

The dosages of the compounds of the invention will depend on a number of factors which will vary from subject to subject. However, it is believed that generally, the daily oral dosage in humans will utilize 0.1 μg-5 mg/kg body weight, preferably from 1 μg-0.5 mg/kg and more preferably about 1 μg-50 μg/kg. The dose regimen will vary, however, depending on the compound and formulation selected, the condition being treated and the judgment of the practitioner. Optimization of dosage, formulation and regimen is routine for practitioners of the art.

Synthesis of the Invention Compounds

The synthesis of the invention compounds is set forth in the above-referenced PCT publication WO00/71535, incorporated herein by reference.

The following compounds of Tables 2 and 3 were prepared and many tested for their ability to inhibit p38-α kinase. It was found that the compounds in Tables 2 and 3 provide IC₅₀ values for inhibition of p38-α in the range of 0.1-1.5 μMol.

TABLE 2

| Compd. # | STRUCTURE | MW (Calcd.) | MW (Obsd.) |
|---|---|---|---|
| 1 | 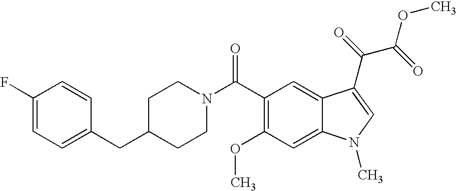 | 466 | 466 |
| 2 | 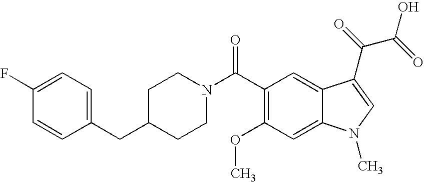 | 452 | 453 |
| 3 | 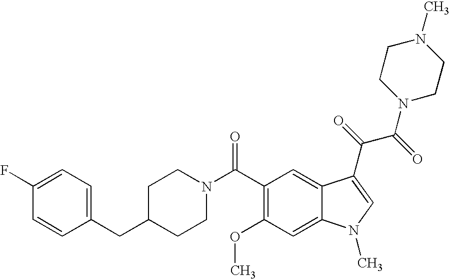 | 535 | 534 |
| 4 | 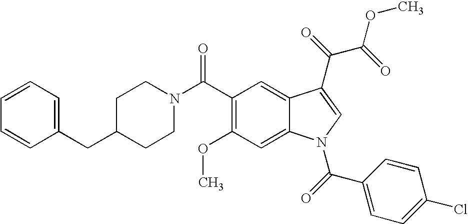 | 573 | 573 |
| 5 | 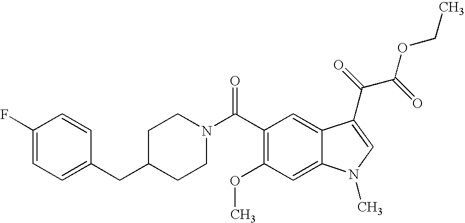 | 480 | 480 |
| 6 | 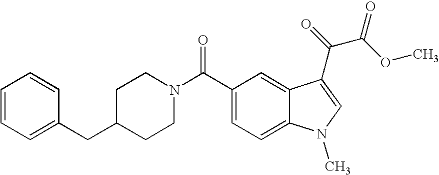 | 418 | 418 |
| 7 | 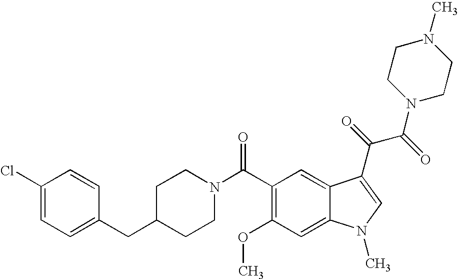 | 551 | 551 |
| 8 | 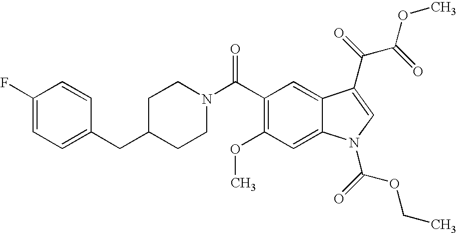 | 524 | 523 |
| 9 | 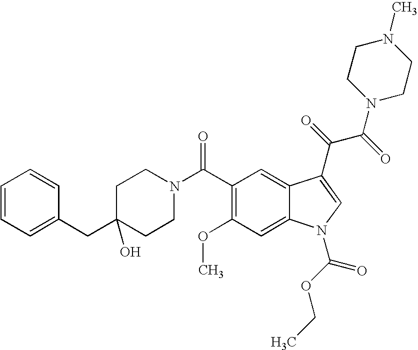 | 590 | 590 |
| 10 | 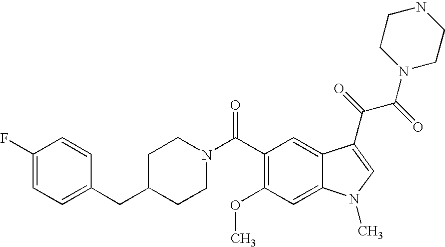 | 521 | 520 |
| 11 | 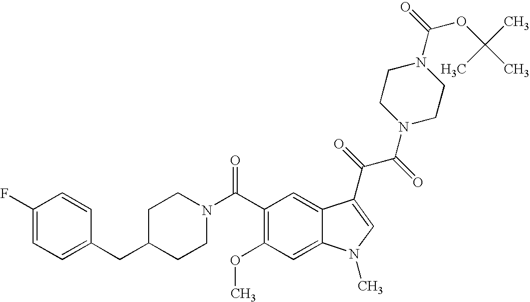 | 620 | 620 |
| 12 | 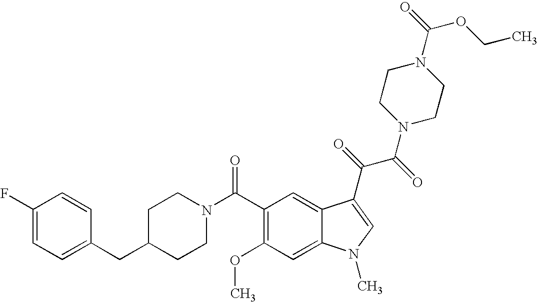 | 592 | 592 |
| 13 | 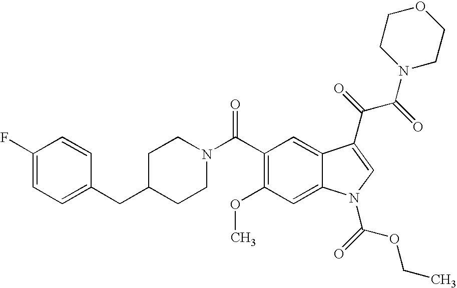 | 579 | 580 |
| 14 | 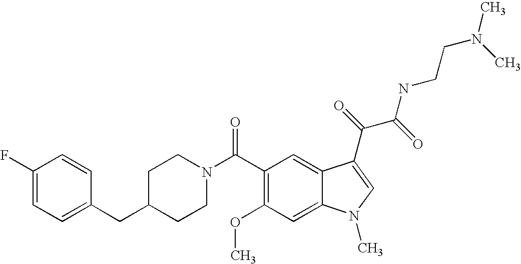 | 523 | 522 |
| 15 | 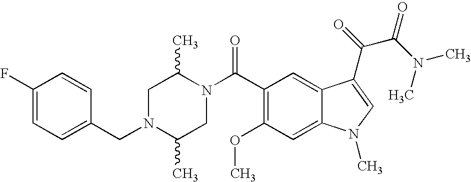 | 509 | 509 |
| 16 | 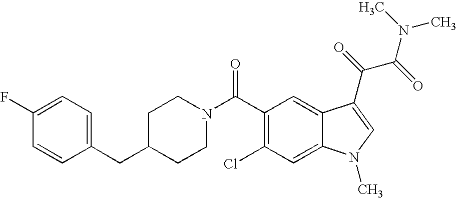 | 484 | 484 |
| 17 | 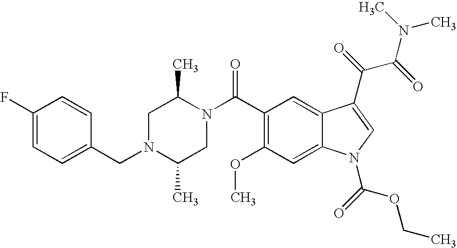 | 567 | 567 |
| 18 | 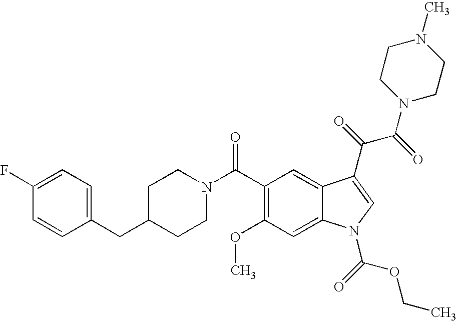 | 593 | 592 |
| 19 | 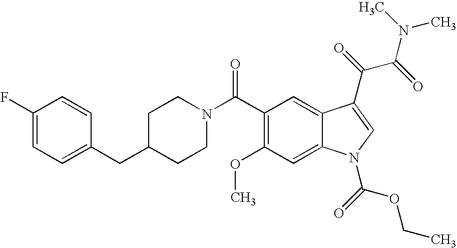 | 537 | 537 |
| 20 | 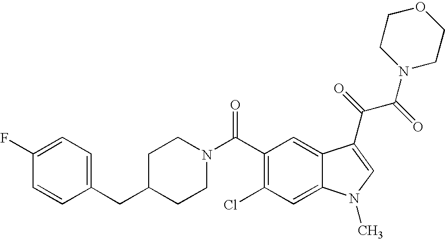 | 526 | 525 |
| 21 | 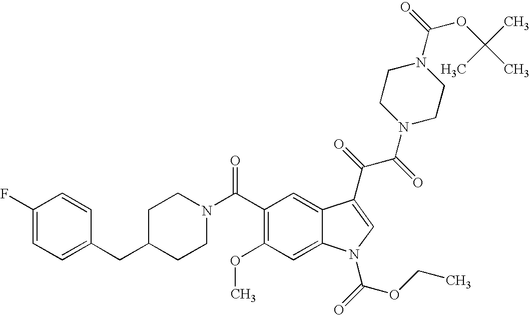 | 678 | 678 |
| 22 | 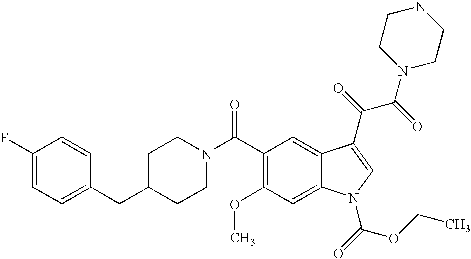 | 579 | 578 |
| 23 | 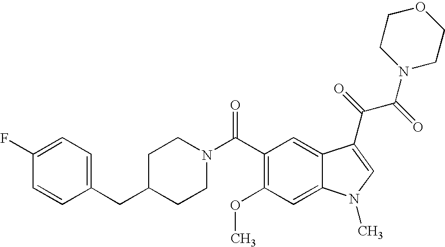 | 522 | 522 |
| 24 | 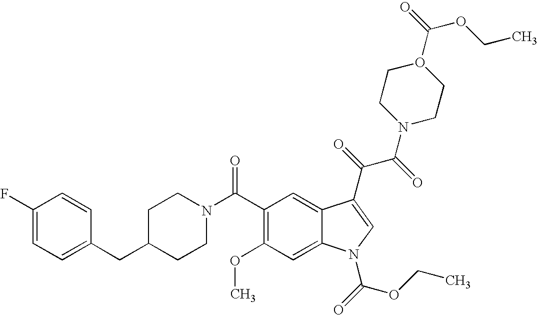 | 650 | 650 |
| 25 | 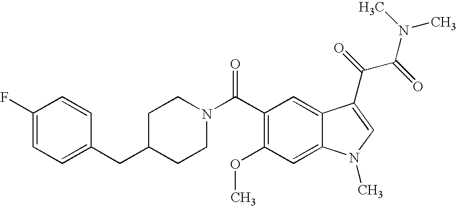 | 480 | 480 |
| 26 | 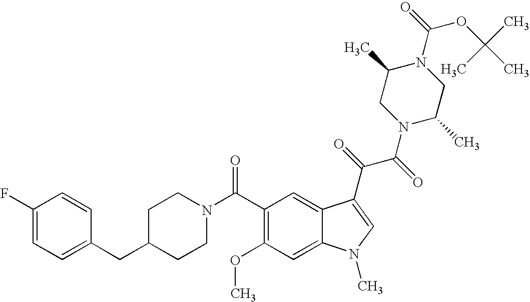 | 648 | 648 |
| 27 | 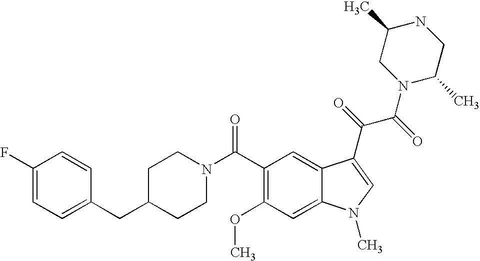 | 549 | 548 |
| 28 | 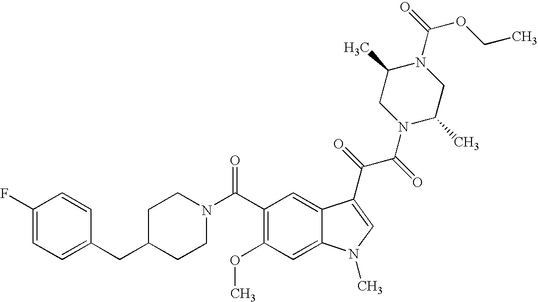 | 620 | 620 |
| 29 | 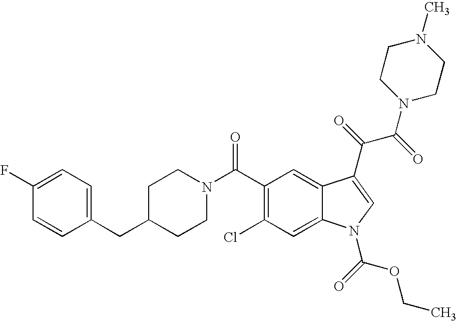 | 597 | 596 |
| 30 | 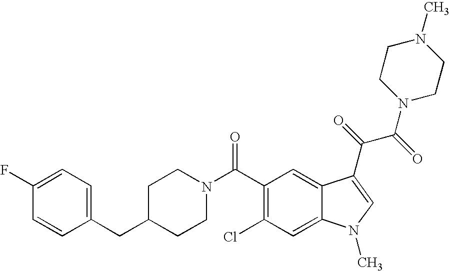 | 539 | 538 |
| 31 | 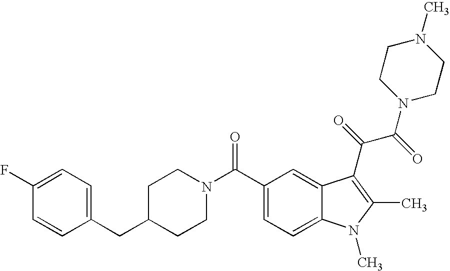 | 519 | 519 |
| 32 | 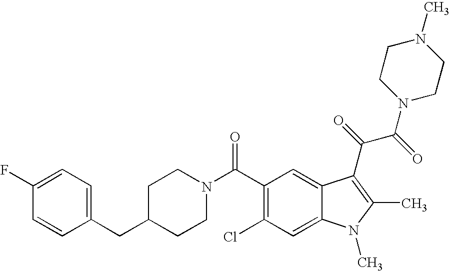 | 553 | 553 |
| 33 | 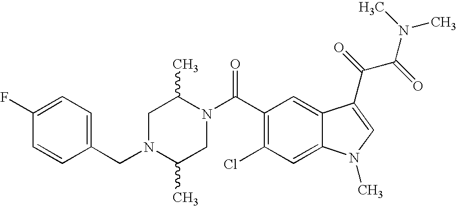 | 513 | 513 |
| 34 | 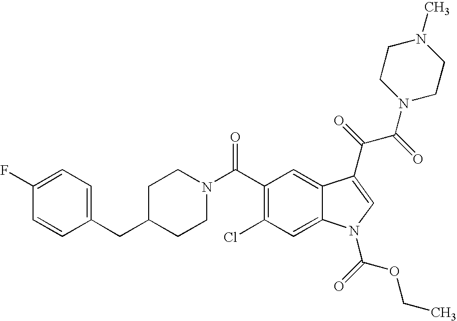 | 609 | 609 |
| 35 | 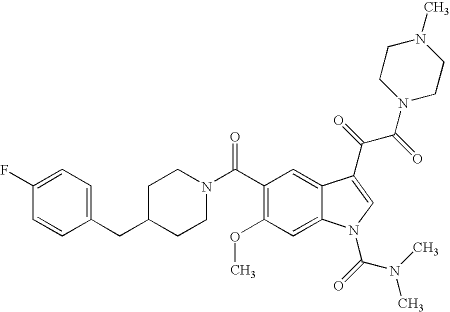 | 592 | 591 |
| 36 | 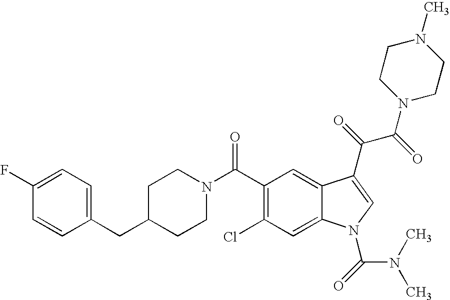 | 596 | 595 |
| 37 | 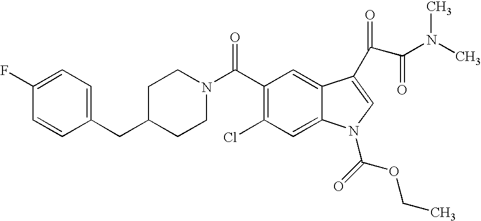 | 542 | 541 |
| 38 | 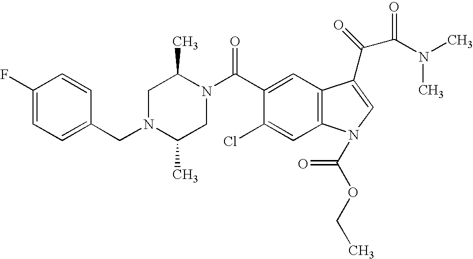 | 571 | 571 |
| 39 | 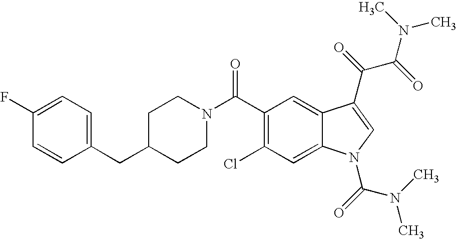 | 541 | 541 |
| 40 | 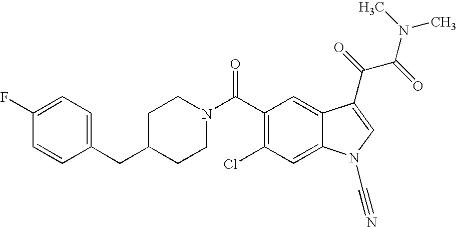 | 494 | 494 |
| 41 | 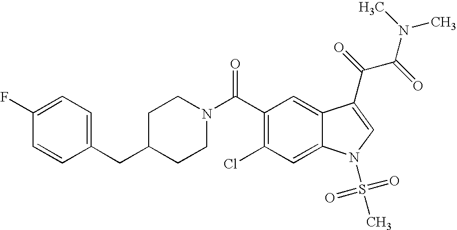 | 548 | 548 |
| 42 | 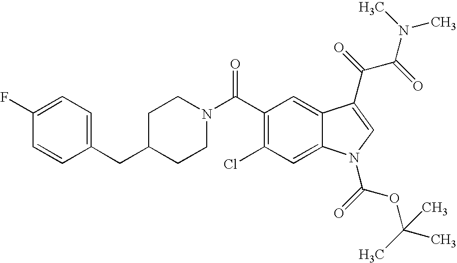 | 570 | 570 |
| 43 | 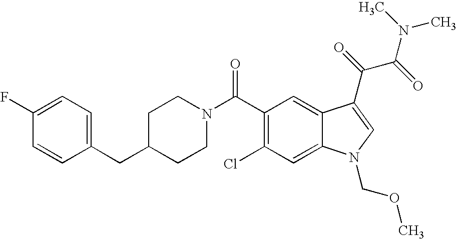 | 514 | 513 |
| 44 | 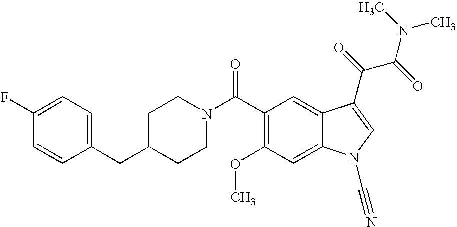 | 490 | 490 |
| 45 | 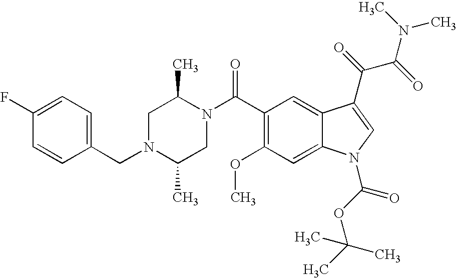 | 595 | 595 |
| 46 | 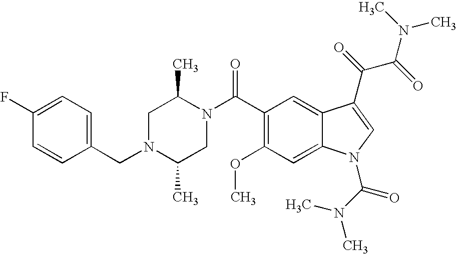 | 566 | 566 |
| 47 | 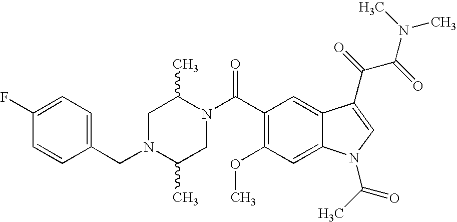 | 537 | 537 |
| 48 | 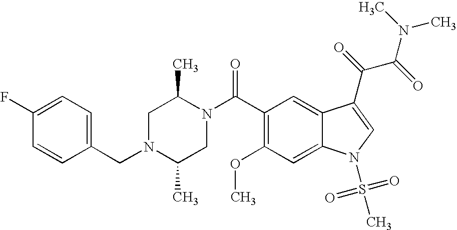 | 573 | 573 |
| 49 | 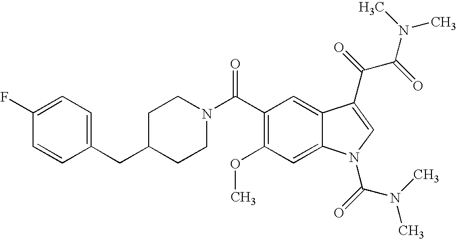 | 536 | 536 |
| 50 | 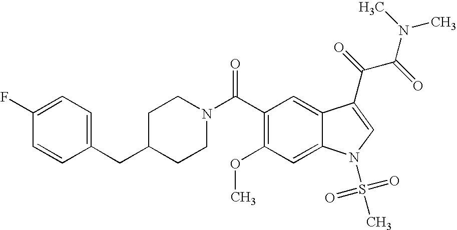 | 543 | 543 |
| 51 | 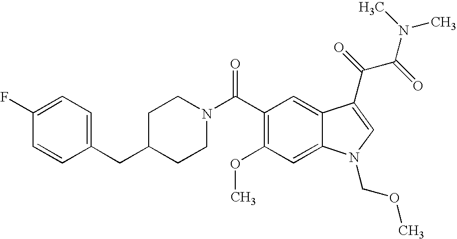 | 509 | 509 |
| 52 | 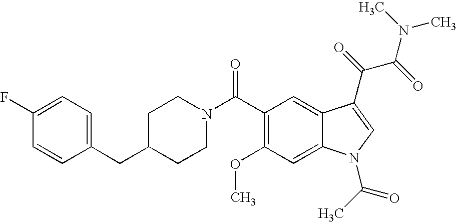 | 507 | 507 |
| 53 | 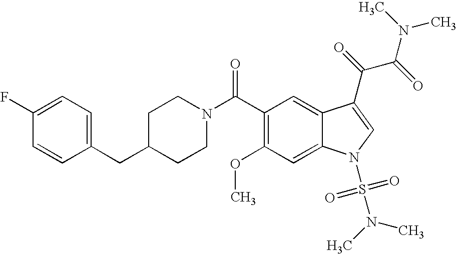 | 572 | 572 |
| 54 | 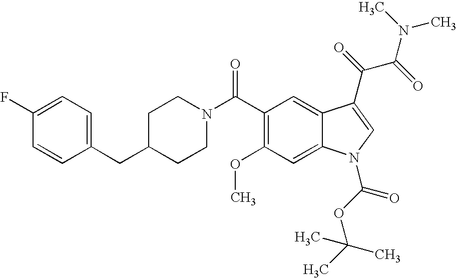 | 565 | 565 |
| 55 | 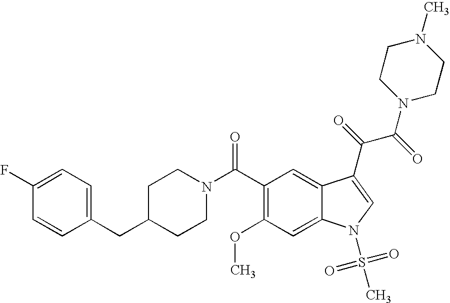 | 599 | 599 |
| 56 | 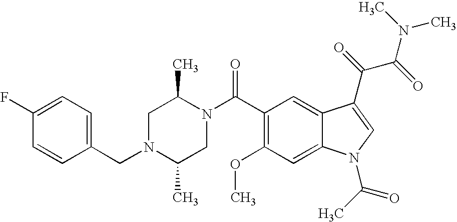 | 537 | 537 |
| 57 | 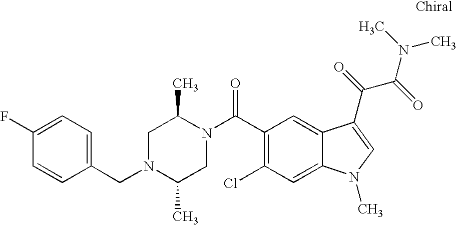 | 513 | 513 |
| 58 | 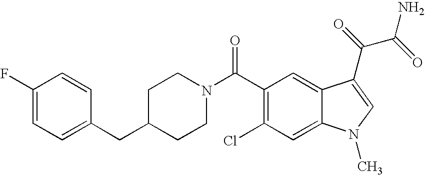 | 456 | 456 |
| 59 | 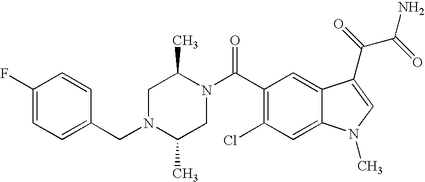 | 485 | 485 |
| 60 | 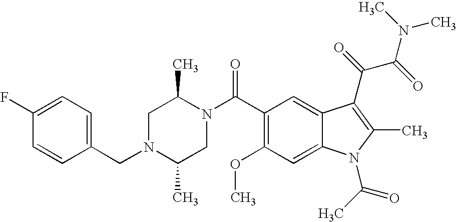 | 551 | 551 |
| 61 | 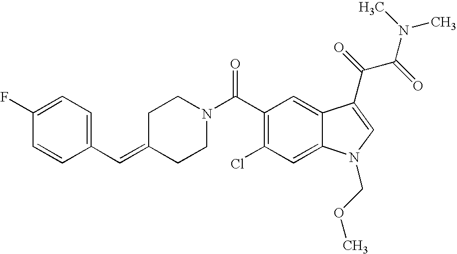 | 511 | 511 |
| 62 | 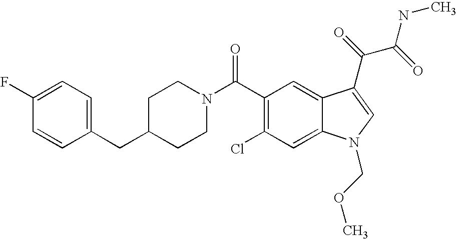 | 499 | 500 |
| 63 | 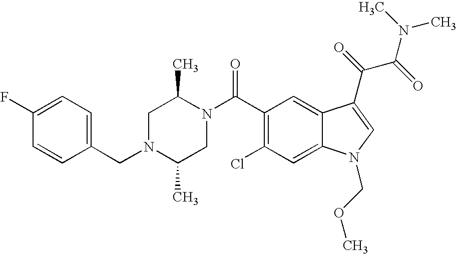 | 543 | 543 |
| 64 | 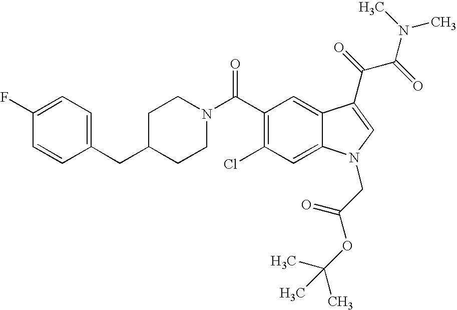 | 584 | 584 |
| 65 | 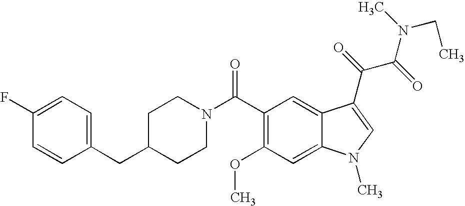 | 493 | 493 |
| 66 | 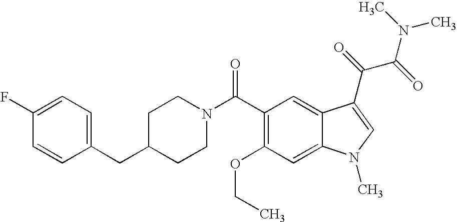 | 494 | 494 |
| 67 | 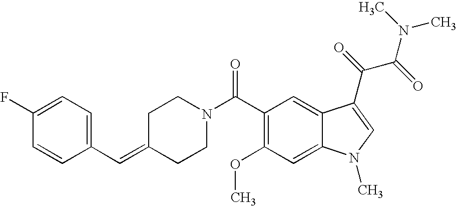 | 477 | 477 |
| 68 | 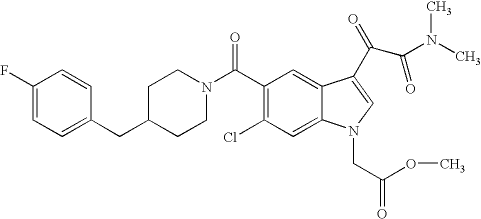 | 542 | 542 |
| 69 | 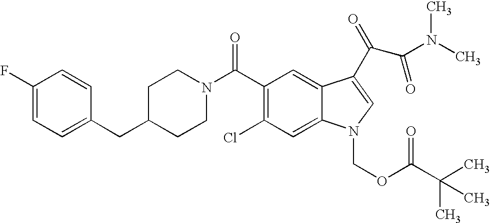 | 584 | 584 |
| 70 | 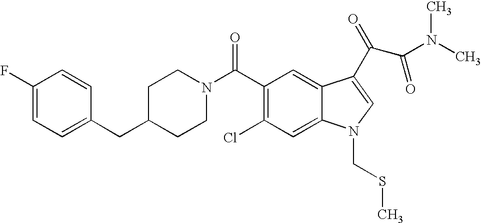 | 530 | 529 |
| 71 | 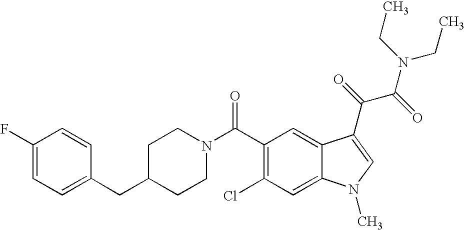 | 512 | 511 |
| 72 | 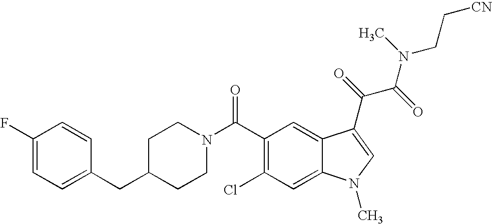 | 523 | 522 |
| 73 | 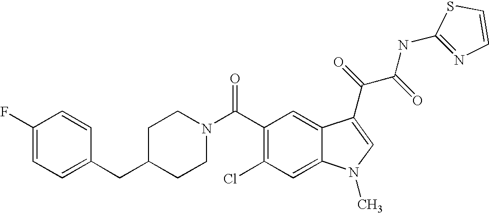 | 539 | 539 |
| 74 | 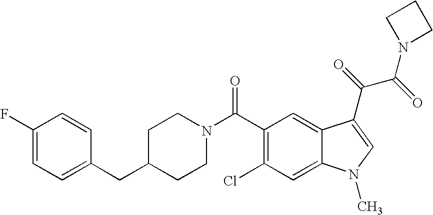 | 495 | 495 |
| 75 | 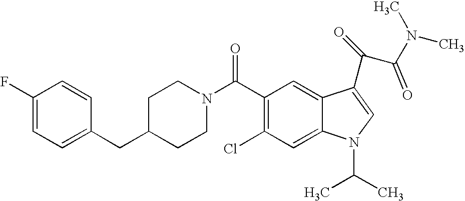 | 512 | 511 |
| 76 | 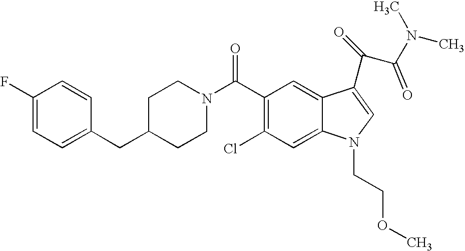 | 528 | 528 |
| 77 | 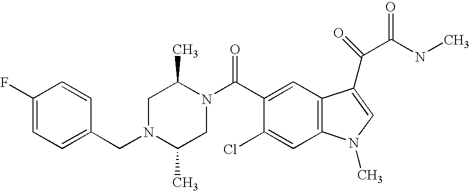 | 499 | 499 |
| 78 | 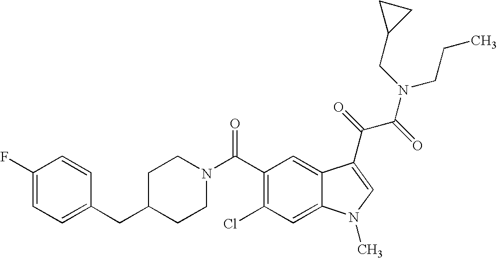 | 552 | 551 |
| 79 | 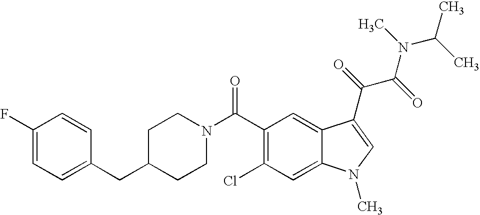 | 512 | 511 |
| 80 | 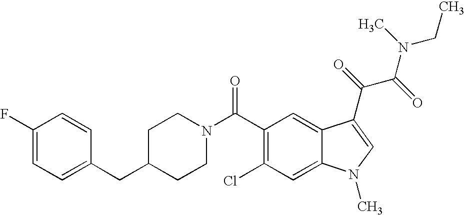 | 498 | 497 |
| 81 | 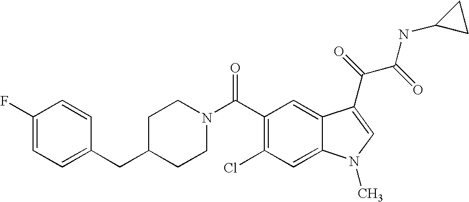 | 496 | 495 |
| 82 | 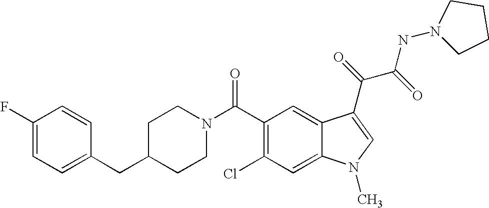 | 525 | 525 |
| 83 | 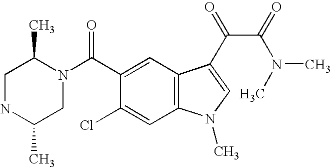 | 405 | 405 |
| 84 | 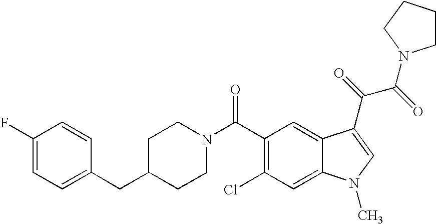 | 510 | 509 |
| 85 | 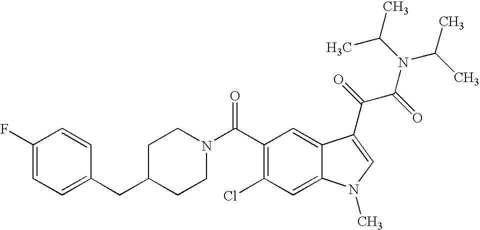 | 540 | 539 |
| 86 | 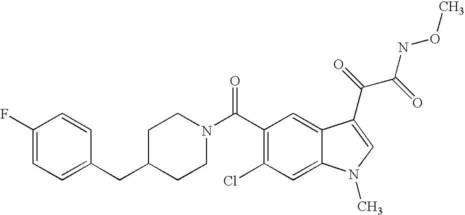 | 485 | 486 |
| 87 | 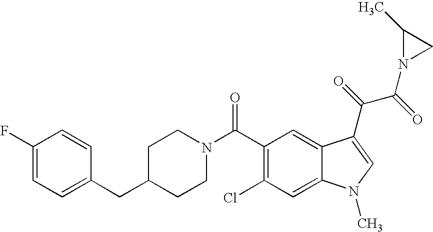 | 495 | 495 |
| 88 | 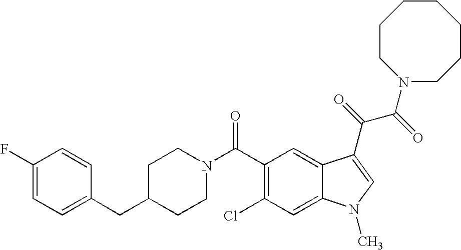 | 552 | 551 |
| 89 | 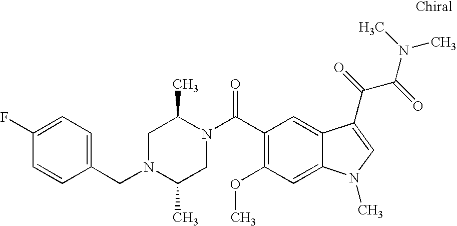 | 508 | 508 |
| 90 | 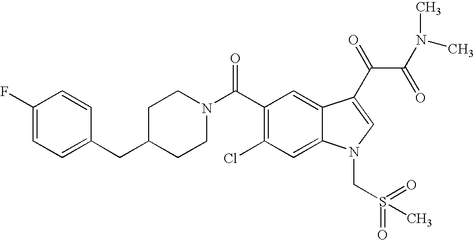 | 562 | 562 |
| 91 | 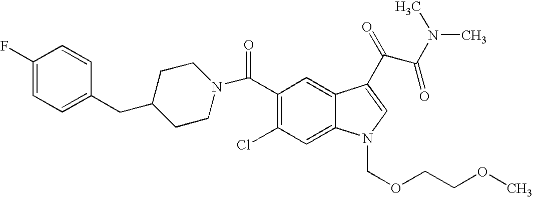 | 558 | 558 |
| 92 | 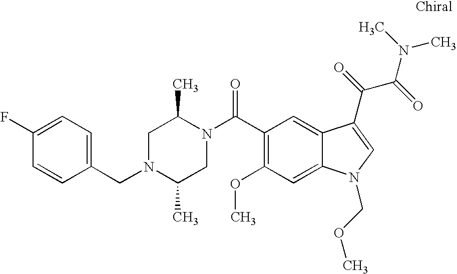 | 539 | 539 |
| 93 | 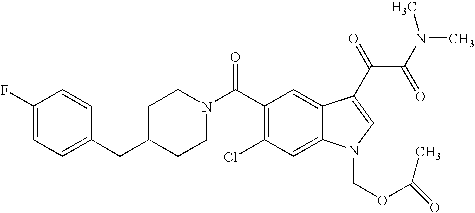 | 542 | 542 |
| 94 | 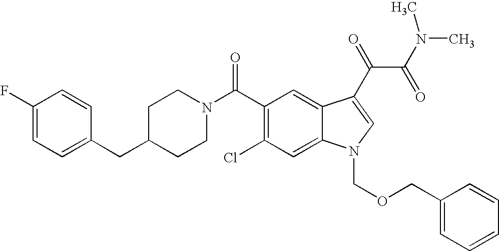 | 590 | 590 |
| 95 | 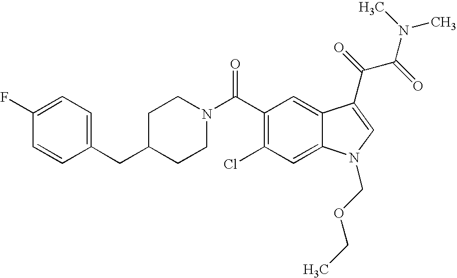 | 528 | 528 |
| 96 | 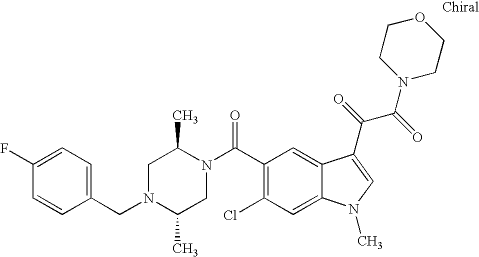 | 555 | 555 |
| 97 | 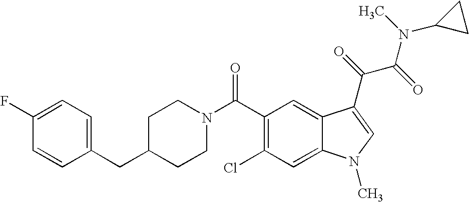 | 510 | 509 |
| 98 | 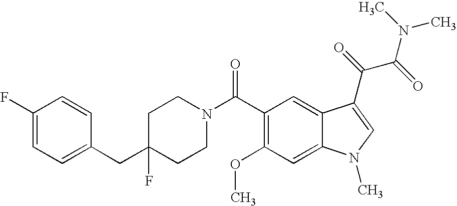 | 497 | 497 |
| 99 | 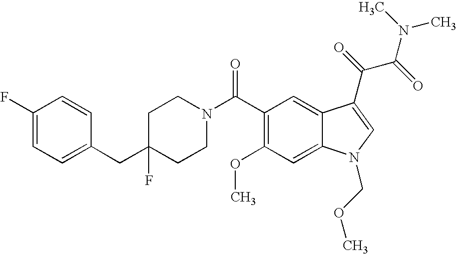 | 527 | 527 |
| 100 | 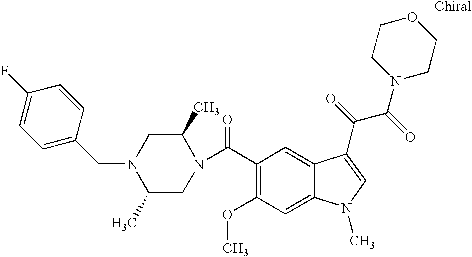 | 550 | 550 |
| 101 | 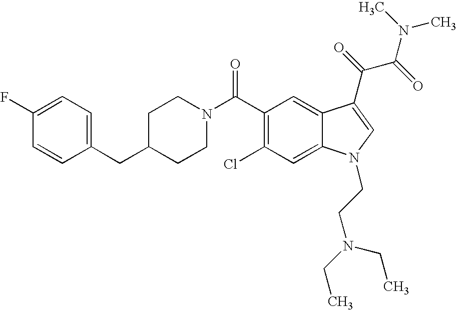 | 569 | 569 |
| 102 | 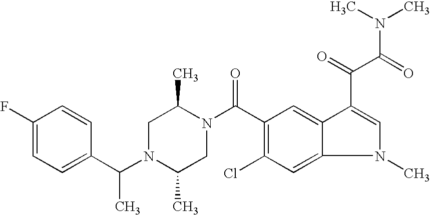 | 527 | 527 |
| 103 | 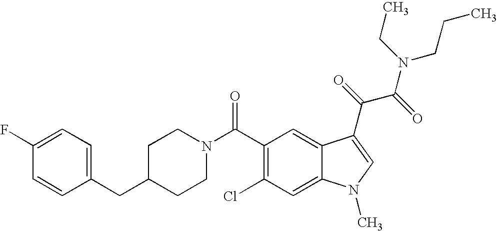 | 526 | 525 |
| 104 | 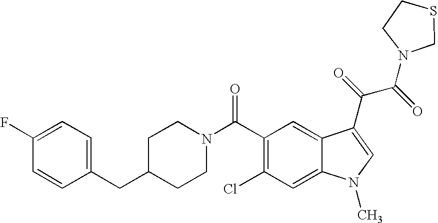 | 528 | 528 |
| 105 | 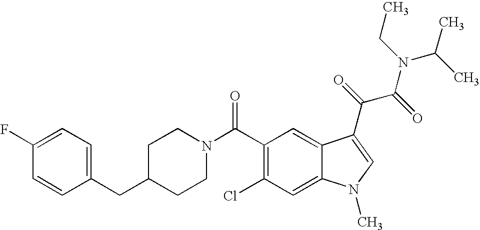 | 526 | 525 |
| 106 | 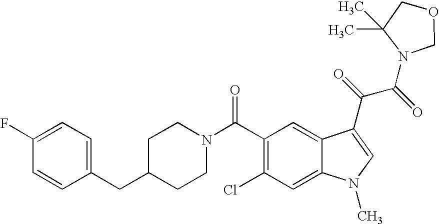 | 540 | 539 |
| 107 | 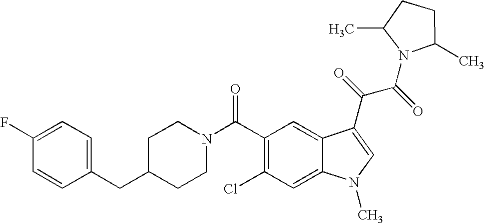 | 538 | 537 |
| 108 | 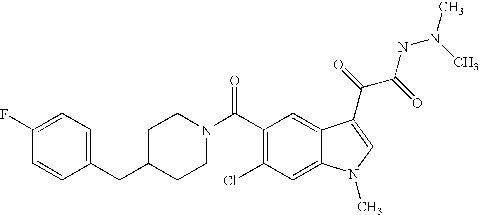 | 498 | 498 |
| 109 | 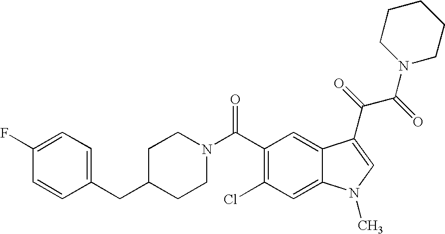 | 524 | 523 |
| 110 | 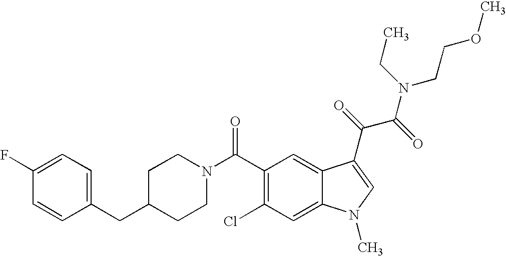 | 542 | 541 |
| 111 | 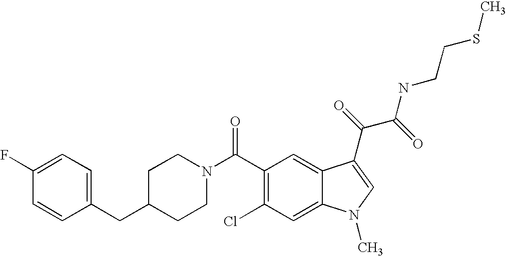 | 530 | 529 |
| 112 | 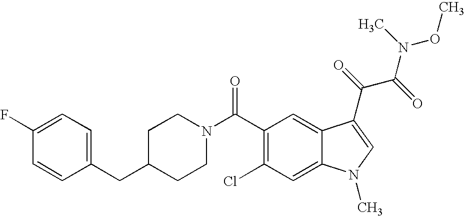 | 499 | 500 |
| 113 | 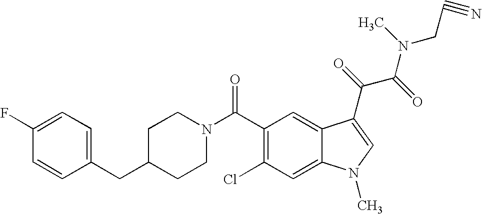 | 508 | 508 |
| 114 | 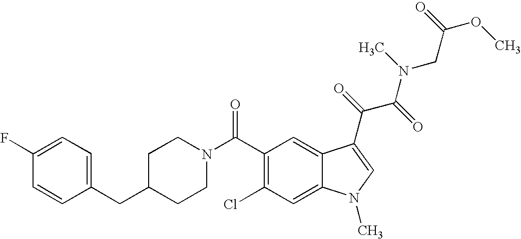 | 542 | 541 |
| 115 | 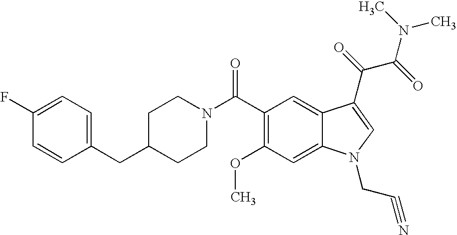 | 504 | 504 |
| 116 | 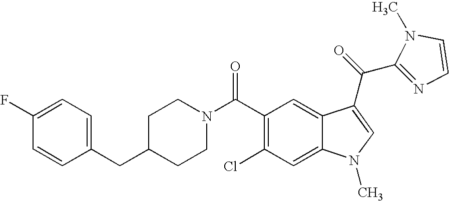 | 492 | 504 |

TABLE 3

| Compd. # | MOLSTRUCTURE | MW (Calcd.) | MW (Obs.) |
|---|---|---|---|
| 117 | 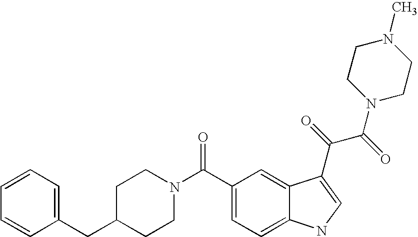 | 472.5858 | 472.5858 |
| 118 | 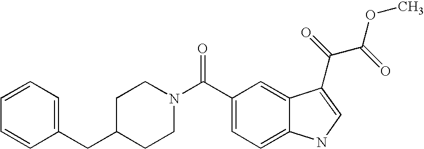 | 404.4636 | 404.4636 |
| 119 | 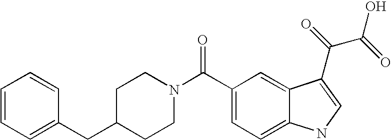 | 390.4368 | 390.4368 |
| 120 | 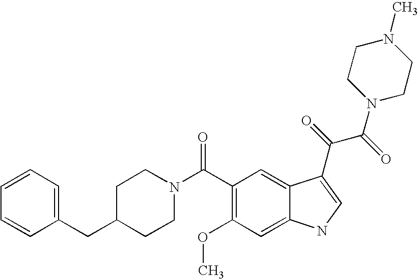 | 502.6116 | 502.6116 |
| 121 | 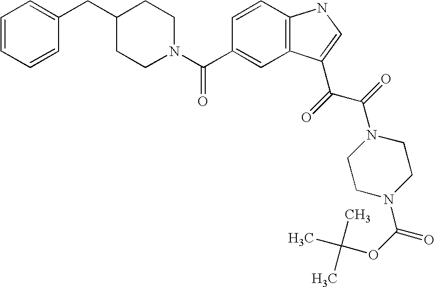 | 558.6752 | 558.6752 |
| 122 | 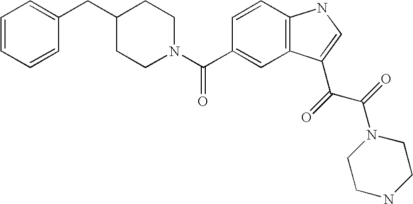 | 458.559 | 458.559 |
| 123 | 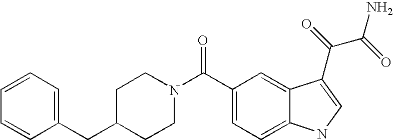 | 389.4527 | 389.4527 |
| 124 | 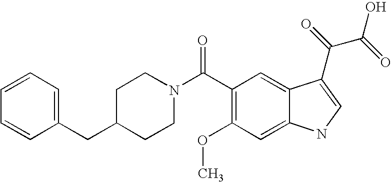 | 420.4626 | 420.4626 |
| 125 | 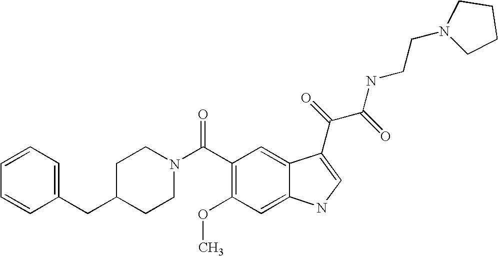 | 516.6384 | 516.6384 |
| 126 | 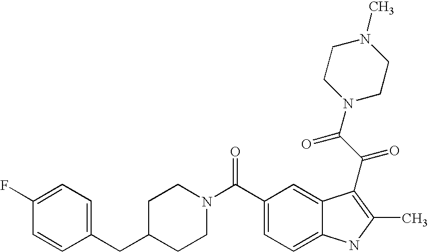 | 504.6027 | 504.6027 |
| 127 | 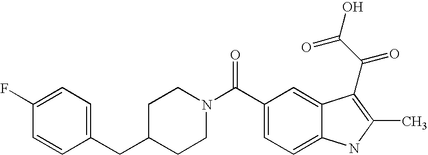 | 422.4537 | 422.4537 |
| 128 | 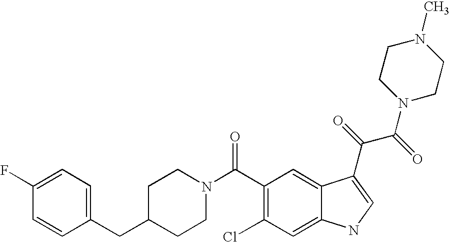 | 525.021 | 525.021 |
| 129 | 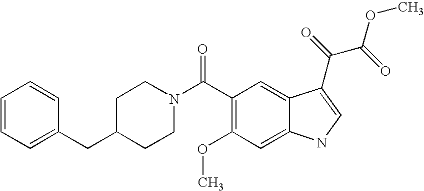 | 434.4894 | 434.4894 |
| 130 | 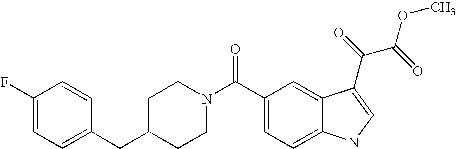 | 422.4537 | 422.4537 |
| 131 | 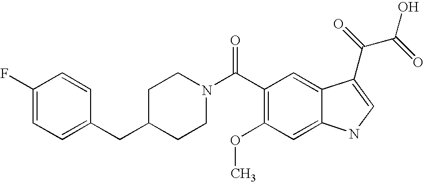 | 438.4527 | 438.4527 |
| 132 | 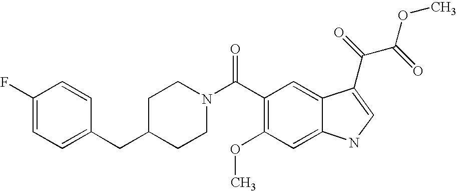 | 452.4795 | 452.4795 |
| 133 | 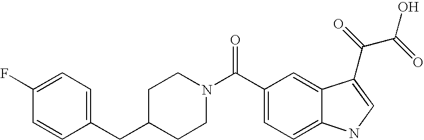 | 408.4269 | 408.4269 |
| 134 | 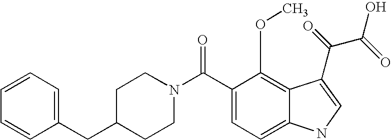 | 420.4626 | 420.4626 |
| 135 | 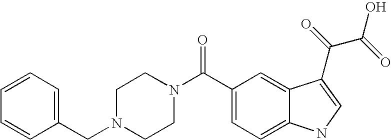 | 391.4249 | 391.4249 |
| 136 | 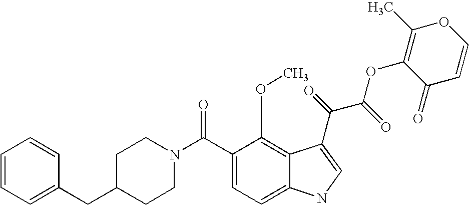 | 528.5582 | 528.5582 |
| 137 | 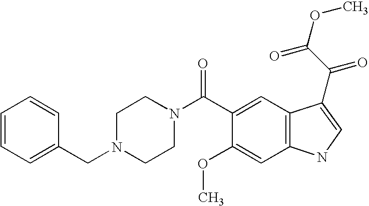 | 435.4775 | 435.4775 |
| 138 | 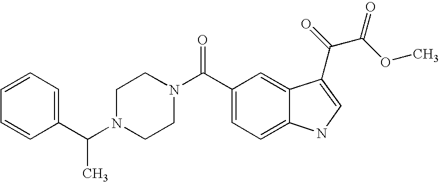 | 419.4785 | 419.4785 |
| 139 | 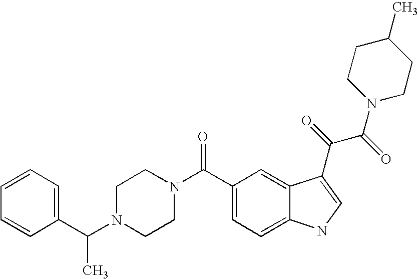 | 486.6126 | 486.6126 |
| 140 | 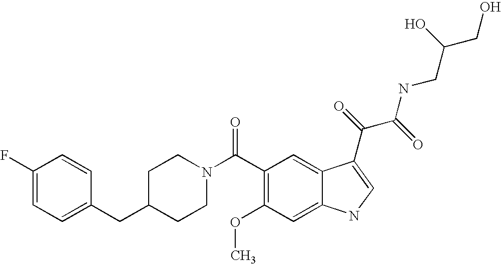 | 511.547 | 511.547 |
| 141 | 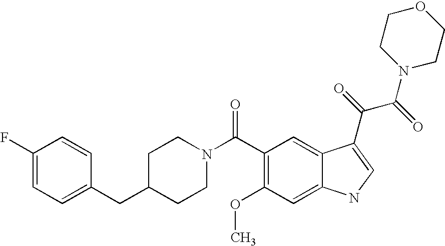 | 507.559 | 507.559 |
| 142 | 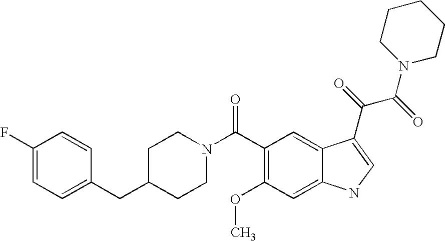 | 505.5868 | 505.5868 |
| 143 | 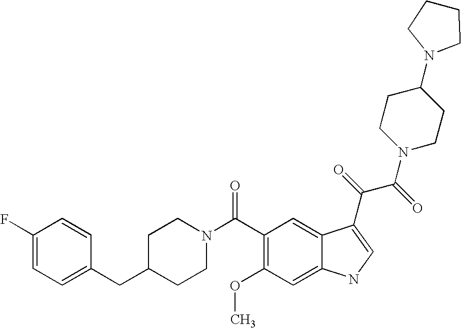 | 574.6931 | 574.6931 |
| 144 | 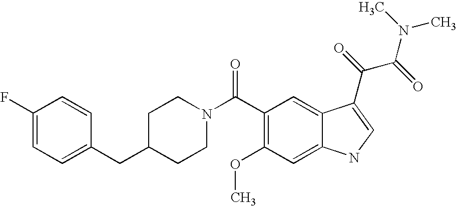 | 465.5222 | 465.5222 |
| 145 | 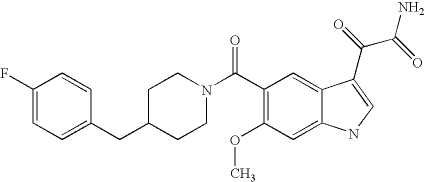 | 437.4686 | 437.4686 |
| 146 | 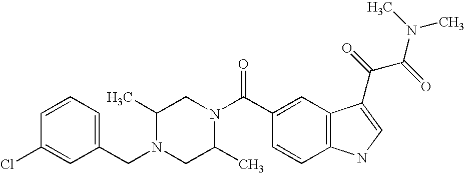 | 480.9931 | 480.9931 |
| 147 | 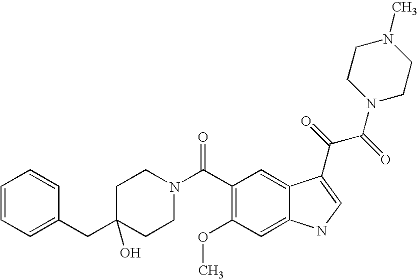 | 518.6106 | 518.6106 |
| 148 | 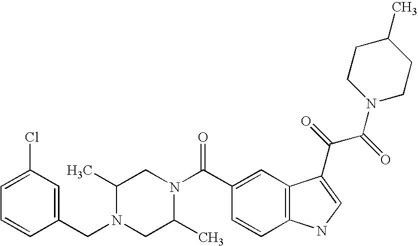 | 535.0845 | 535.0845 |
| 149 | 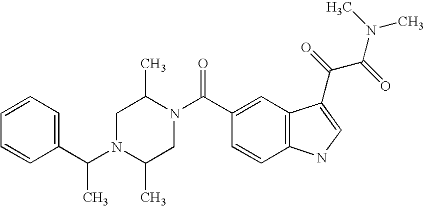 | 460.5748 | 460.5748 |
| 150 | 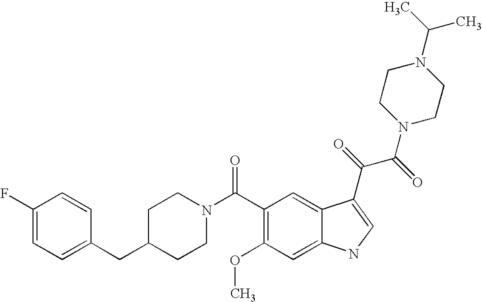 | 548.6553 | 548.6553 |
| 151 | 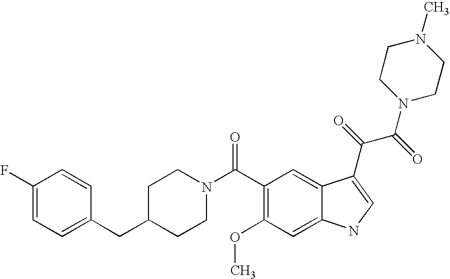 | 520.6017 | 520.6017 |
| 152 | 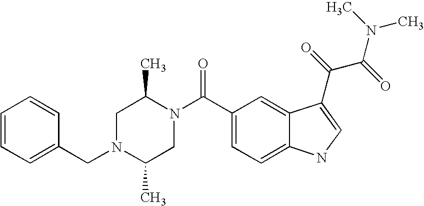 | 446.548 | 446.548 |
| 153 | 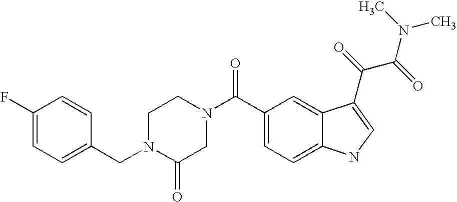 | 450.4677 | 450.4677 |
| 154 | 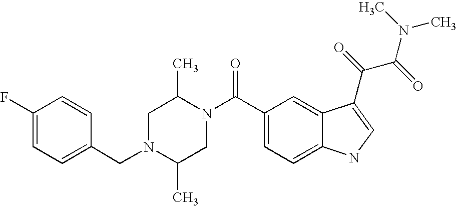 | 494.5639 | 494.5639 |
| 155 | 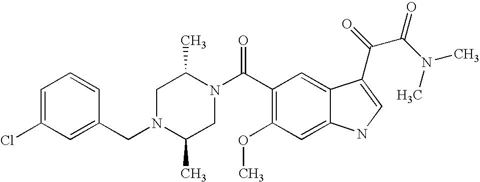 | 511.0189 | 511.0189 |
| 156 | 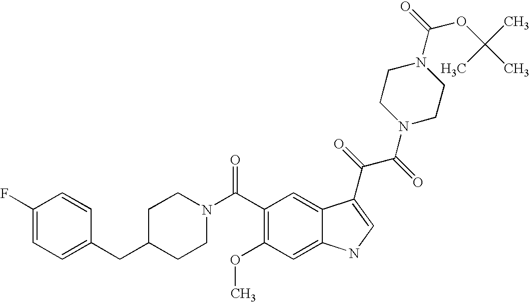 | 606.6911 | 606.6911 |
| 157 | 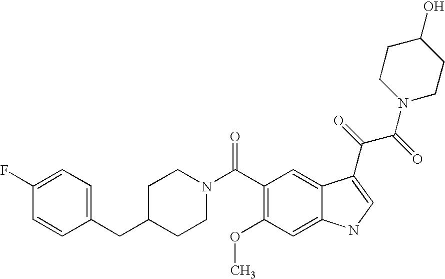 | 521.5858 | 521.5858 |
| 158 | 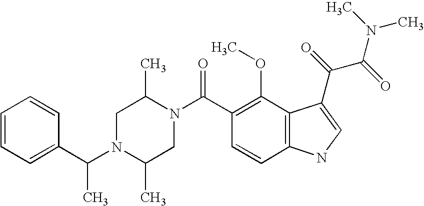 | 490.6006 | 490.6006 |
| 159 | 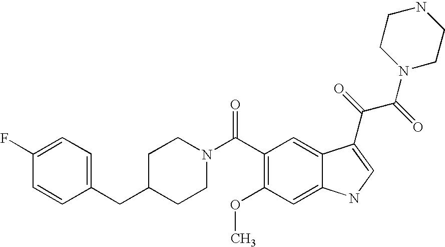 | 506.5749 | 506.5749 |
| 160 | 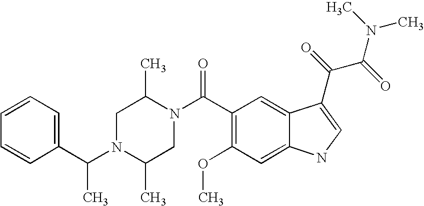 | 490.6006 | 490.6006 |
| 161 | 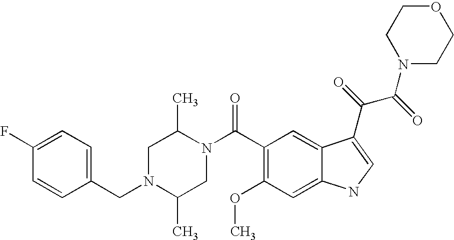 | 536.6007 | 536.6007 |
| 162 | 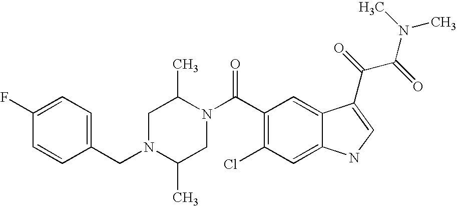 | 498.9832 | 498.9832 |
| 163 | 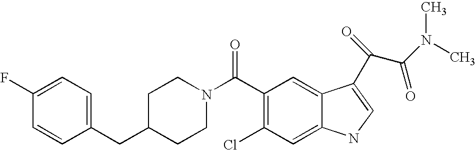 | 469.9415 | 469.9415 |
| 164 | 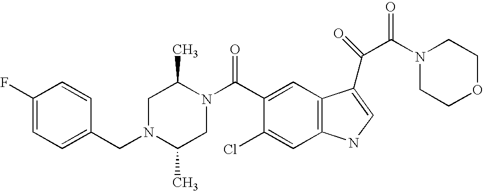 | 541.02 | 541.02 |
| 165 | 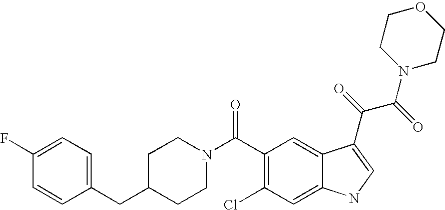 | 511.9783 | 511.9783 |
| 166 | 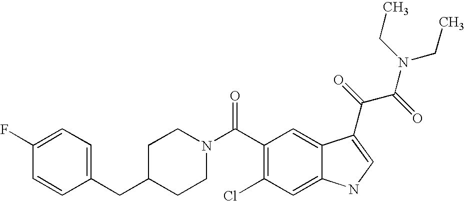 | 497.9951 | 497.9951 |
| 167 | 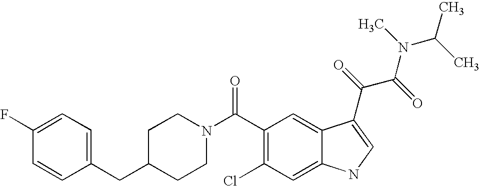 | 497.9951 | 497.9951 |
| 168 | 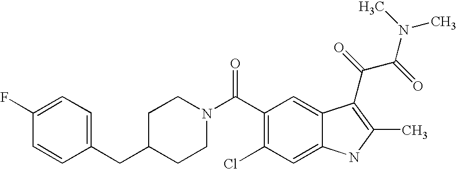 | 483.9683 | 483.9683 |
| 169 | 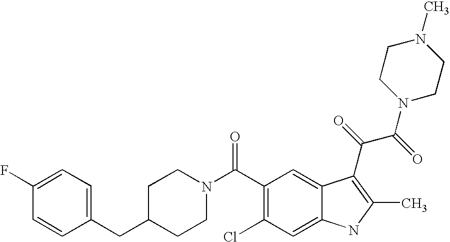 | 539.0478 | 539.0478 |
| 170 | 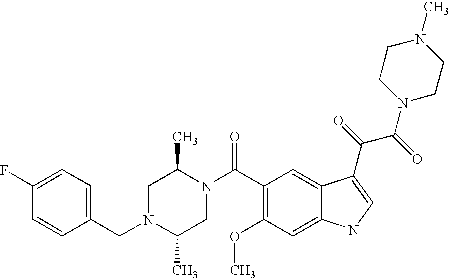 | 549.6434 | 549.6434 |
| 171 | 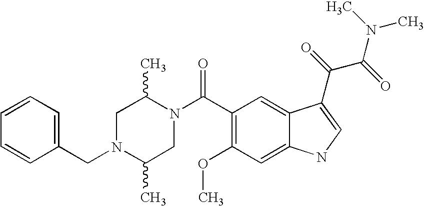 | 476.5738 | 476.5738 |
| 172 | 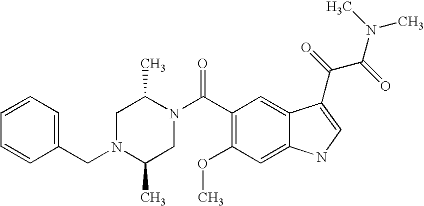 | 476.5738 | 476.5738 |
| 173 | 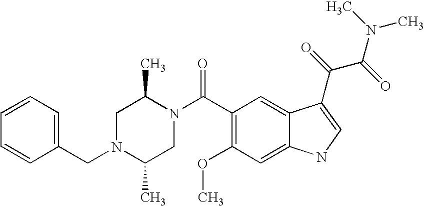 | 476.5738 | 476.5738 |
| 174 | 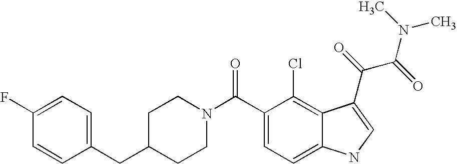 | 469.9415 | 469.9415 |
| 175 | 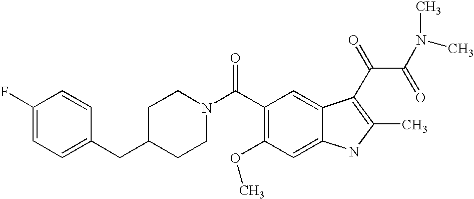 | 479.549 | 479.549 |
| 176 | 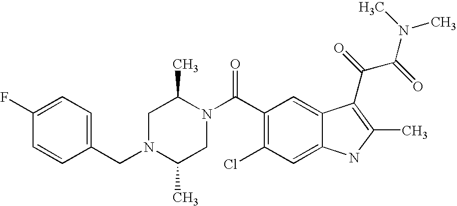 | 513.01 | 513.01 |
| 177 | 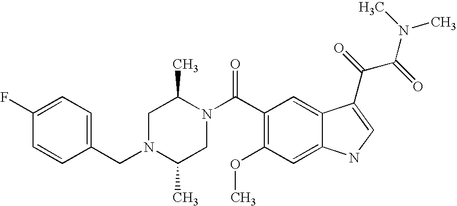 | 494.5639 | 494.5639 |
| 178 | 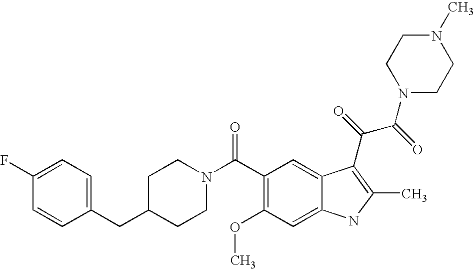 | 534.6285 | 534.6285 |
| 179 | 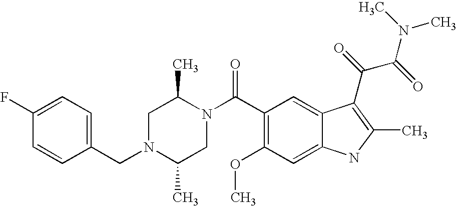 | 508.5907 | 508.5907 |
| 180 | 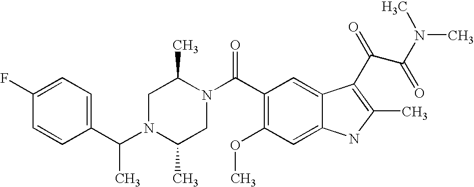 | 522.6175 | 522.6175 |
| 181 | 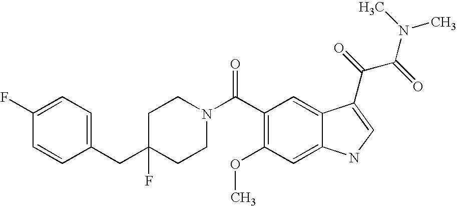 | 483.5123 | 483.5123 |

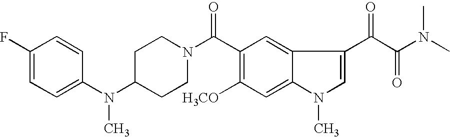

1. A method to treat cystic fibrosis in a human subject which method comprises administering to a subject in need of such treatment a therapeutically effective amount of a compound of the formula:
or pharmaceutically acceptable salts thereof, a prodrug form thereof or a pharmaceutical composition thereof, wherein [symbol]
represents a single or double bond;
one Z² is CA or CR⁸A and the other is CR¹, CR¹₂, NR⁶ or N wherein each R¹, R⁶ and R⁸ is independently hydrogen or noninterfering substituent;
A is -Wᵢ-COXⱼY wherein Y is COR² or an isostere thereof and R² is hydrogen or a noninterfering substituent, each of W and X is a spacer of 2-6 Å, and each of i and j is independently 0 or 1;
Z³ is NR⁷ or 0;
R⁷ is a noninterfering substituent;
each R³ is independently a noninterfering substituent;
n is 0-3;
each of L¹ and L² is a linker;
each R⁴ is independently a noninterfering substituent;
m is 0-4;
Z¹ is CR⁵ or N wherein R⁵ is hydrogen or a noninterfering substituent;
each of l and k is an integer from 0-2 wherein the sum of l and k is 0-3;
Ar is an aryl group substituted with 0-5 noninterfering substituents, wherein two noninterfering substituents can form a fused ring; and
the distance between the atom of Ar linked to L² and the center of the α ring is 4.5-24 Å.

2. The method of claim 1 wherein A is COXⱼCOR², and
wherein R² is H, or is straight or branched chain alkyl, alkenyl, alkynyl, aryl, arylalkyl, heteroalkyl, heteroaryl, or heteroarylalkyl, each optionally substituted with halo, alkyl, heteroalkyl, SR, OR, NR₂, OCOR, NRCOR, NRCONR₂, NRSO₂R, NRSO₂NR₂ OCONR₂, CN, COOR, CONR₂, COR, or R₃Si wherein each R is independently H, alkyl, alkenyl or aryl or the heteroatom-containing forms thereof, or wherein R² is OR, NR₂, SR, NRCONR₂, OCONR₂, or NRSO₂NR₂, wherein each R is independently H, alkyl, alkenyl or aryl or the heteroatom-containing forms thereof, and wherein two R attached to the same atom may form a 3-8 member ring and wherein said ring may further be substituted by alkyl, alkenyl, alkynyl, aryl, arylalkyl, heteroalkyl, heteroaryl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroatom-containing forms thereof wherein two R attached to the same atom may form a 3-8 member ring, optionally substituted as above defined; and
X, if present, is alkylene.

3. The method of claim 1 wherein Y is an isostere of COR².

4. The method of claim 3 wherein Y is tetrazole; 1,2,3-triazole; 1,2,4-triazole; or imidazole.

5. The method of claim 1 wherein each of i and j is 0.

6. The method of claim 2 wherein j is 0.

7. The method of claim 1 wherein Z³ is NR⁷.

8. The method of claim 7 wherein R⁷ is H or is optionally substituted alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof.

9. The method of claim 8 wherein R⁷ is H, or is optionally substituted alkyl, or acyl.

10. The method of claim 1 wherein both k and l are 1.

11. The method of claim 1 wherein L¹ is CO, CHOH or CH₂.

12. The method of claim 11 wherein L¹ is CO.

13. The method of claim 1 wherein Z¹ is N.

14. The method of claim 1 wherein Z¹ is CR⁵ wherein R⁵ is H, OR, NR₂, SR or halo, wherein each R is independently H, alkyl, alkenyl or aryl or the heteroatom-containing forms thereof,

15. The method of claim 1 wherein L² is alkylene (1-4C) or alkenylene (1-4C) optionally substituted with a moiety selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety.

16. The method of claim 15 wherein L² is unsubstituted alkylene.

17. The method of claim 15 wherein L² is unsubstituted methylene, methylene substituted with alkyl, or —CH═.

18. The method of claim 1 wherein Ar is optionally substituted with 0-5 substituents selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two of said optional substituents on adjacent positions can be joined to form a fused, optionally substituted aromatic or nonaromatic, saturated or unsaturated ring which contains 3-8 members.

19. The method of claim 18 wherein Ar is optionally substituted phenyl.

20. The method of claim 19 wherein said optional substitution is by halo, OR, or alkyl.

21. The method of claim 20 wherein said phenyl is unsubstituted or has a single substituent.

22. The method of claim 1 wherein R⁴ is selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof.

23. The method of claim 22 wherein each R⁴ is halo, OR, or alkyl.

24. The method of claim 23 wherein m is 0, 1, or 2.

25. The method of claim 24 wherein m is 2 and both R⁴ are alkyl.

26. The method of claim 1 wherein each R³ is halo, alkyl, heteroalkyl, OCOR, OR, NRCOR, SR, or NR₂, wherein R is H, alkyl, aryl, or heteroforms thereof.

27. The method of claim 26 wherein R³ is halo or alkoxy.

28. The method of claim 27 wherein n is 0, 1 or 2.

29. The method of claim 1 wherein L¹ is coupled to the a ring at the 4-, 5- or 6-position.

30. The method of claim 1 wherein Z² at position 3 is CA or CH¹A.

31. The method of claim 30 wherein the Z² at position 2 is CR¹ or CR¹_{2.}

32. The method of claim 31 wherein R¹ is hydrogen, or is alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R¹ can be joined to form a fused, optionally substituted aromatic or nonaromatic, saturated or unsaturated ring which contains 3-8 members.

33. The method of claim 32 wherein each R¹ is selected from the group consisting of H, alkyl, acyl, aryl, arylalkyl, heteroalkyl, heteroaryl, halo, OR, NR₂, SR, NRCOR, alkyl-OOR, RCO, COOR, and CN, wherein each R is independently H, alkyl, or aryl or heteroforms thereof.

34. The method of claim 30 wherein Z² at position 2 is N or NR⁶.

35. The method of claim 34 wherein R⁶ is H, or alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, or R₃Si wherein each R is independently H, alkyl, alkenyl or aryl or heteroforms thereof.

36. The method of claim 1 wherein [symbol] represents a double bond.

37. The method of claim 1 wherein the distance between the atom on Ar linked to L² and the center of the a ring is 7.5-11 Å.

38. The method of claim 1 wherein the compound of formula (1) is selected from the group consisting of compounds shown in Tables 2 and 3 herein.

39. A pharmaceutical composition for treating cystic fibrosis in a human subject which composition comprises
a therapeutically effective amount of a compound of or mixtures of compounds of claim 1 in admixture with at least one pharmaceutically acceptable excipient.

40. The composition of claim 39 which further contains an additional therapeutic agent.
